(19) United States
(12) Patent Application Publication
(10) Pub. No.: US 2024/0153491 A1
(43) Pub. Date: May 9, 2024

(54) SYSTEMS AND METHODS FOR ENHANCING AUDIO COMMUNICATIONS

(71) Applicant: AVAIL MEDSYSTEMS, INC., Santa Clara, CA (US)

(72) Inventors: Daniel HAWKINS, Palo Alto, CA (US); Ravi KALLURI, San Jose, CA (US); Shivakumar MAHADEVAPPA, Fremont, CA (US)

(21) Appl. No.: 18/327,375

(22) Filed: Jun. 1, 2023

Related U.S. Application Data
(63) Continuation of application No. PCT/US21/61859, filed on Dec. 3, 2021.
(60) Provisional application No. 63/121,655, filed on Dec. 4, 2020.

(51) Int. Cl. G10L 15/02; G06V 10/25; G10L 15/08; G10L 17/14

(52) U.S. Cl. CPC G10L 15/02; G06V 10/25; G10L 15/083; G10L 17/14; G10L 2015/088

(57) ABSTRACT

The present disclosure provides systems and methods for enhancing audio communications. In one aspect, the present disclosure provides a method for enhancing audio communications. The method may comprise (a) detecting one or more parameters associated with a medical procedure and one or more audio communications associated with the medical procedure; and (b) processing the one or more audio communications based on the one or more parameters to generate one or more enhanced audio communications.

### CROSS-REFERENCE

This application is a continuation of International Patent Application PCT/US21/61859, filed on Dec. 3, 2021, which claims priority to U.S. Provisional Application No. 63/121,655 filed on Dec. 4, 2020, each of which is incorporated herein by reference in its entirety for all purposes.

### BACKGROUND

Medical practitioners may perform various procedures within a medical suite, such as an operating room. Often times, the operating room may be occupied by a plurality of medical practitioners, or persons other than a medical practitioner, such as medical staff. During a medical procedure, many individuals may be talking or communicating simultaneously and concurrently. This may hinder coordination and/or communications between the individuals in the operating room.

### SUMMARY

Recognized herein are various limitations with audio and video based systems and methods for monitoring, supporting, and performing medical operations. The present disclosure provides systems and methods for enhancing the quality of audio communications made in relation to a surgical procedure or medical operation. The systems and methods of the present disclosure may be implemented to detect and/or recognize tools, products, and/or individuals based on the voices or the voice activity of such individuals. In some cases, the systems and methods of the present disclosure may be implemented to prioritize audio communications made by one or more persons of interest, based on an identity of a speaker or a content of the audio communication made by the speaker. In some cases, the systems and methods of the present disclosure may be implemented to focus a detection of one or more audio communications using beam forming and related methods for adjusting a directionality or directivity of one or more audio detection devices.

In one aspect, the present disclosure provides a method for enhancing audio communications. The method may comprise (a) detecting one or more parameters associated with a medical procedure and one or more audio communications associated with the medical procedure; and (b) processing the one or more audio communications based on the one or more parameters to generate one or more enhanced audio communications.

In some embodiments, the one or more parameters comprise a physical feature, a face, a voice, or an identity of a human or a robot that made the one or more audio communications. In some embodiments, the one or more parameters comprise a key word, phrase, or sentence of the one or more audio communications. In some embodiments, the one or more parameters comprise a type of tool or instrument in use or a phase of the medical procedure.

In some embodiments, processing the one or more audio communications comprises beam forming to adjust a detection area, a detection range, a directivity, or a directionality of one or more audio detection devices. In some embodiments, processing the one or more audio communications comprises prioritizing a detection or a capture of the one or more audio communications based on an identity of a speaker. In some embodiments, processing the one or more audio communications comprises adjusting the priority of detection or capture based on a detection of one or more key words, phrases, or sentences in the one or more audio communications. In some embodiments, processing the one or more audio communications comprises adjusting the priority of detection or capture based on a detection of one or more key words, phrases, or sentences in the one or more audio communications. In some embodiments, processing the one or more audio communications comprises increasing a volume of a first audio communication of the one or more audio communications relative to a volume of a second audio communication of the one or more audio communications. In some embodiments, processing the one or more audio communications comprises decreasing a volume of a first audio communication of the one or more audio communications relative to a volume of a second audio communication of the one or more audio communications. In some embodiments, processing the one or more audio communications comprises muting or eliminating one or more audio communications.

In some embodiments, the one or more enhanced audio communications correspond to a tool or instrument of interest or a usage of the tool or instrument of interest. In some embodiments, the one or more enhanced audio communications correspond to a surgical phase of interest. In some embodiments, the one or more enhanced audio communications correspond to a doctor, a surgeon, a medical worker, a vendor representative, or a product specialist of interest.

In some embodiments, the method may further comprise detecting the one or more parameters using computer vision, natural language processing, or machine learning. In some embodiments, detecting the one or more parameters comprises identifying a medical tool or instrument that is associated with the one or more audio communications. In some embodiments, identifying the medical tool or instrument comprises imaging the tool or instrument, scanning a identifier associated with the tool or instrument, or receiving one or more electromagnetic waves comprising information on the tool or instrument.

In another aspect, the present disclosure provides a method for enhancing audio communications, comprising: (a) receiving a plurality of audio communications associated with a medical procedure; (b) receiving one or more user inputs corresponding to a parameter of interest, wherein the parameter of interest is associated with a performance of one or more steps of the medical procedure; and (c) generating one or more enhanced audio communications based on the plurality of audio communications and the one or more user inputs. In some embodiments, the one or more user inputs comprise a user selection of the parameter of interest. In some embodiments, the parameter of interest comprises an instrument, a specialist, a representative, a doctor, a surgeon, or a surgical phase of interest. In some embodiments, the one or more user inputs comprise a selection of an audio channel of interest from a master list of audio channels of interest.

In some embodiments, generating the one or more enhanced audio communications comprises isolating or extracting one or more audio channels associated with the parameter of interest. In some embodiments, generating the one or more enhanced audio communications comprises increasing a volume of a first audio communication of the plurality of audio communications relative to a volume of a second audio communication of the plurality of audio communications. In some embodiments, generating the one or more enhanced audio communications comprises decreasing a volume of a first audio communication of the plurality of audio communications relative to a volume of a second audio communication of the plurality of audio communications. In some embodiments, generating the one or more enhanced audio communications comprises muting or eliminating one or more audio communications.

In some embodiments, the one or more enhanced audio communications are generated by post-processing one or more videos associated with the medical procedure to isolate, extract, or augment one or more audio channels associated with parameter of interest. In some embodiments, the one or more enhanced audio communications are generated based on metadata associated with plurality of audio communications or one or more videos of the medical procedure. In some embodiments, the one or more enhanced audio communications correspond to a plurality of audio channels. In some embodiments, the plurality of audio channels correspond to a plurality of doctors, surgeons, vendor representatives, or product specialists supporting the medical procedure. In some embodiments, the plurality of audio channels correspond to a plurality of different tools used to perform one or more steps of the medical procedure. In some embodiments, the plurality of audio channels correspond to a plurality of different steps or phases of the medical procedure.

In some embodiments, processing the one or more audio communications comprises (i) enhancing one or more audio communications or (ii) muting or eliminating one or more audio communications for one or more users. In some embodiments, the one or more audio communications are processed by a broadcaster, a moderating entity, a remote specialist, a vendor representative, or the one or more users, wherein the one or more users comprise at least one user viewing a surgical video or a portion thereof.

In some embodiments, the method may further comprise using one or more cameras or imaging sensors to track a field of view for an area from which the plurality of audio communications are received or captured. In some embodiments, the method may further comprise transmitting the field of view to one or more remote participants. In some embodiments, one or more audio beams or regions of interest are selectable by the one or more remote participants, wherein the one or more audio beams or regions of interest correspond to (i) at least a subset of the plurality of audio communications or (ii) one or more regions within the field of view. In some embodiments, the selection of the one or more audio beams or regions of interest is performed locally or remotely.

In some embodiments, the method may further comprise tracking or tagging one or more individuals or regions of interest. In some embodiments, the method may further comprise selecting (i) a set of audio signals to enhance or (ii) a set of audio signals to remove or attenuate. In some embodiments, the method may further comprise tracking the one or more individuals or regions of interest as the one or more individuals move relative to the one or more cameras or imaging sensors. In some embodiments, the selection of audio beams or regions of interest is pre-registered before the medical procedure starts. In some embodiments, the selection of audio beams or regions of interest is made for recorded content associated with the medical procedure.

In another aspect, the present disclosure provides a method for processing audio communications, comprising: (a) receiving a plurality of audio communications from one or more individuals associated with or performing a medical procedure; and (b) detecting, recognizing, or identifying one or more tools, products, or instruments associated with the medical procedure based on at least a subset of the plurality of audio communications from the one or more individuals. In some embodiments, (a) comprises using one or more microphones or a microphone array comprising the one or more microphones to receive the plurality of audio communications. In some embodiments, the one or more microphones are configured to detect one or more keywords within the plurality of audio communications or a subset thereof. In some embodiments, the one or more tools, products, or instruments are identified based on the one or more keywords. In some embodiments, the one or more tools, products, or instruments are identified using natural language processing. In some embodiments, the natural language processing is implemented using one or more algorithms for analyzing the plurality of audio communications.

In some embodiments, the one or more algorithms are configured to implement context aware natural language processing to (i) interpret the plurality of audio communications and (ii) determine which tools or products are being used to perform the medical procedure. In some embodiments, the one or more algorithms are configured to implement context aware natural language processing to (i) interpret the plurality of audio communications and (ii) determine which tools or products are being requested by a doctor or a surgeon performing the medical procedure. In some embodiments, the one or more algorithms are configured to implement context aware natural language processing to (i) interpret the plurality of audio communications and (ii) determine what kind of procedure is being performed or what step of the procedure is being performed. In some embodiments, the one or more algorithms are configured to implement context aware natural language processing to (i) interpret the plurality of audio communications and (ii) catalog (a) different steps in the procedure, (b) a timing of one or more steps of the procedure, or (c) which tools or products are used by a doctor or a hospital to perform the medical procedure. In some embodiments, the one or more algorithms are configured to use natural language processing on the plurality of audio communications to generate or compile data on a timing of steps in a surgical procedure or a volume or frequency of usage for the tools, products, or instruments. In some embodiments, the one or more algorithms are configured to use natural language processing on the plurality of audio communications to determine success rates and/or failure rates for different procedures or procedural steps that are identified using the natural language processing. In some embodiments, the one or more algorithms are configured to use natural language processing on the plurality of audio communications to determine success rates and/or failure rates for different procedures that are performed using the tools, products, or instruments that are identified using the natural language processing.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates an audio capture system that may be utilized within a medical suite to monitor, capture, and enhance audio communications.

FIG. 2 schematically illustrates a plurality of audio recording devices that may be used to capture one or more audio communications, in accordance with some embodiments.

FIG. 3 schematically illustrates an example of a priority list that may be used to prioritize detection of audio communications, in accordance with some embodiments.

FIG. 4 schematically illustrates one or more beams that may be generated for an audio detection device, in accordance with some embodiments.

FIG. 5 schematically illustrates, in accordance with some embodiments an exemplary system for detecting and enhancing audio communications, in accordance with some embodiments.

FIG. 6 schematically illustrates a computer system that is programmed or otherwise configured to implement methods provided herein.

FIG. 7 schematically illustrates a plurality of audio sources that are associated with a plurality of audio channels, in accordance with some embodiments.

FIG. 8 schematically illustrates a selection of one or more audio channels of interest by a user, in accordance with some embodiments.

FIG. 9 schematically illustrates an example of a user interface for selecting one or more audio sources or audio channels of interest from a plurality of audio sources or audio channels, in accordance with some embodiments.

FIG. 10 schematically illustrates an audio management system for post-processing of a plurality of audio sources or channels to provide a customized or tailored selection of audio channels to various users, in accordance with some embodiments.

FIG. 11 schematically illustrates an audio management system that is configured to adjust which audio channels are provisioned to a user, based on one or more inputs provided by the user, in accordance with some embodiments.

FIG. 12 schematically illustrates an exemplary user interface for selecting various audio channels of interest, in accordance with some embodiments.

FIG. 13 schematically illustrates a broadcaster configured to broadcast one or more audio channels, in accordance with some embodiments.

FIG. 14 schematically illustrates a moderating entity configured to selectively enhance or mute various audio channels for certain users or viewers, in accordance with some embodiments.

FIG. 15 schematically illustrates an example of a first user modifying one or more audio channels for a second user, in accordance with some embodiments.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “real time” or “real-time,” as used interchangeably herein, generally refers to an event (e.g., an operation, a process, a method, a technique, a computation, a calculation, an analysis, a visualization, an optimization, etc.) that is performed using recently obtained (e.g., collected or received) data. In some cases, a real time event may be performed almost immediately or within a short enough time span, such as within at least 0.0001 millisecond (ms), 0.0005 ms, 0.001 ms, 0.005 ms, 0.01 ms, 0.05 ms, 0.1 ms, 0.5 ms, 1 ms, 5 ms, 0.01 seconds, 0.05 seconds, 0.1 seconds, 0.5 seconds, 1 second, or more. In some cases, a real time event may be performed almost immediately or within a short enough time span, such as within at most 1 second, 0.5 seconds, 0.1 seconds, 0.05 seconds, 0.01 seconds, 5 ms, 1 ms, 0.5 ms, 0.1 ms, 0.05 ms, 0.01 ms, 0.005 ms, 0.001 ms, 0.0005 ms, 0.0001 ms, or less.

In an aspect, the present disclosure provides a system for monitoring and enhancing audio communications made during a surgical procedure. Monitoring audio communications, as referred to herein, may comprise using an audio recording device or an audio detection device (e.g., a microphone or an array of microphones) to record and/or detect audio communications made by one or more persons or objects before, during, and/or after a surgical procedure. In some cases, monitoring audio communications may comprise using an audio recording device or an audio detection device (e.g., a microphone or an array of microphones) to identify one or more persons or objects based on audio communications made by the one or more persons or objects. Enhancing audio communications, as referred to and described herein, may comprise improving a transmission quality of an audio communication, increasing a signal to noise ratio for one or more portions of an audio communication, and/or augmenting an audio communication with additional data or information. In some cases, enhancing audio communications may comprise prioritizing one or more portions of an audio communication relative to other portions of the audio communication, or prioritizing one or more audio communications relative to a plurality of audio communications. In some cases, enhancing audio communications may comprise adjusting a detection range, a detection area, a directionality, and/or a directivity of one or more audio detection devices, based on a content of an audio communication or an identity of a source of an audio communication. In some cases, enhancing audio communications may comprise adjusting a sensitivity of one or more audio detection devices to audio communications received from a certain area or region, or from a certain speaker or source.

The systems and methods of the present disclosure may be used to detect and enhance audio communications made during a surgical procedure. As used herein, a surgical procedure may comprise a medical operation on a human or an animal. The medical operation may comprise one or more operations on an internal or external region of a human body or an animal. The medical operation may be performed using at least one or more medical products, medical tools, or medical instruments. Medical products, which may be interchangeably referred to herein as medical tools or medical instruments, may include devices that are used alone or in combination with other devices for therapeutic or diagnostic purposes. Medical products may be medical devices. Medical products may include any products that are used during an operation to perform the operation or facilitate the performance of the operation. Medical products may include tools, instruments, implants, prostheses, disposables, or any other apparatus, appliance, software, or materials that may be intended by the manufacturer to be used for human beings. Medical products may be used for diagnosis, monitoring, treatment, alleviation, or compensation for an injury or handicap. Medical products may be used for diagnosis, prevention, monitoring, treatment, or alleviation of disease. In some instances, medical products may be used for investigation, replacement, or modification of anatomy or of a physiological process. Some examples of medical products may range from surgical instruments (e.g., handheld or robotic), catheters, endoscopes, stents, pacemakers, artificial joints, spine stabilizers, disposable gloves, gauze, IV fluids, drugs, and so forth.

Examples of different types of surgical procedures may include but are not limited to thoracic surgery, orthopedic surgery, neurosurgery, ophthalmological surgery, plastic and reconstructive surgery, vascular surgery, hernia surgery, head and neck surgery, hand surgery, endocrine surgery, colon and rectal surgery, breast surgery, urologic surgery, gynecological surgery, and other types of surgery. In some cases, surgical procedures may comprise two or more medical operations involving a donor and a recipient. In such cases, the surgical procedures may comprise two or more concurrent medical operations to exchange biological material (e.g., organs, tissues, cells, etc.) between a donor and a recipient.

The systems and methods of the present disclosure may be implemented to detect and enhance audio communications made during a surgical procedure conducted in a health care facility. As used herein, a health care facility may refer to any type of facility, establishment, or organization that may provide some level of health care or assistance. In some examples, health care facilities may include hospitals, clinics, urgent care facilities, out-patient facilities, ambulatory surgical centers, nursing homes, hospice care, home care, rehabilitation centers, laboratory, imaging center, veterinary clinics, or any other types of facility that may provide care or assistance. A health care facility may or may not be provided primarily for short term care, or for long-term care. A health care facility may be open at all days and times, or may have limited hours during which it is open. A health care facility may or may not include specialized equipment to help deliver care. Care may be provided to individuals with chronic or acute conditions. A health care facility may employ the use of one or more health care providers (a.k.a. medical personnel/medical practitioner). Any description herein of a health care facility may refer to a hospital or any other type of health care facility, and vice versa.

In some cases, the health care facility may have one or more locations internal to the health care facility where one or more surgical operations may be performed. In some cases, the one or more locations may comprise one or more operating rooms. In some cases, the one or more operating rooms may only be accessible by qualified or approved individuals. Qualified or approved individuals may comprise individuals such as a medical patient or a medical subject undergoing a surgical procedure, medical operators performing one or more steps of a surgical procedure, and/or medical personnel or support staff who are supporting one or more aspects of the surgical procedure. For example, the medical personnel or support staff may be present in an operating room in order to help the medical operators perform one or more steps of the surgical procedure.

The systems and methods of the present disclosure may be implemented using one or more audio recording or audio detection devices. As used herein, an audio recording device may comprise a device that is capable of receiving, recording, and/or detecting audio communications. The one or more audio recording devices may be configured to obtain a plurality of audio communications associated with a surgical procedure. In some cases, the plurality of audio communications may be captured using a plurality of audio recording devices. The plurality of audio recording devices may comprise 1, 2, 3, 4, 5, 6, 7, 8, 9, 10, or more audio recording devices. The plurality of audio recording devices may comprise n audio recording devices, where n is any integer that is greater than or equal to 2.

The plurality of audio recording devices may be provided in different positions and/or orientations relative to a medical subject or medical personnel performing a surgical operation on the medical subject. The plurality of audio recording devices may be provided in a plurality of different positions and/or orientations relative to a medical patient or subject undergoing a medical operation or a medical operator performing a medical operation. The plurality of audio recording devices may be provided in a plurality of different positions and/or orientations relative to each other.

In some cases, the plurality of audio recording devices may be attached to a ceiling, a wall, a floor, a structural element of an operating room (e.g., a beam), an operating table, a medical instrument, or a portion of a medical operator's body (e.g., the medical operator's hand, arm, or head). In some cases, the plurality of audio recording devices may be releasably coupled to a ceiling, a wall, a floor, a structural element of an operating room, an operating table, a medical instrument, or a portion of a medical operator's body.

In some cases, the plurality of audio recording devices may be movable relative to a surface or structural element on which the plurality of audio recording devices are attached, fixed, or releasably coupled. For example, the plurality of audio recording devices may be repositioned and/or rotated to adjust a detection area of the plurality of audio recording devices. In some cases, one or more joints, hinges, arms, rails, and/or tracks may be used to adjust a position and/or an orientation of the plurality of audio recording devices. In some cases, the position and/or the orientation of each of the plurality of audio recording devices may be manually adjustable by a human operator. In other cases, the position and/or the orientation of each of the plurality of audio recording devices may be automatically adjustable in part based on computer-implemented tracking software (e.g., video tracking software and/or audio tracking software). The position and/or the orientation of each of the plurality of audio recording devices may be physically adjusted. The position and/or the orientation of each of the plurality of audio recording devices may be adjusted or controlled remotely by a human operator.

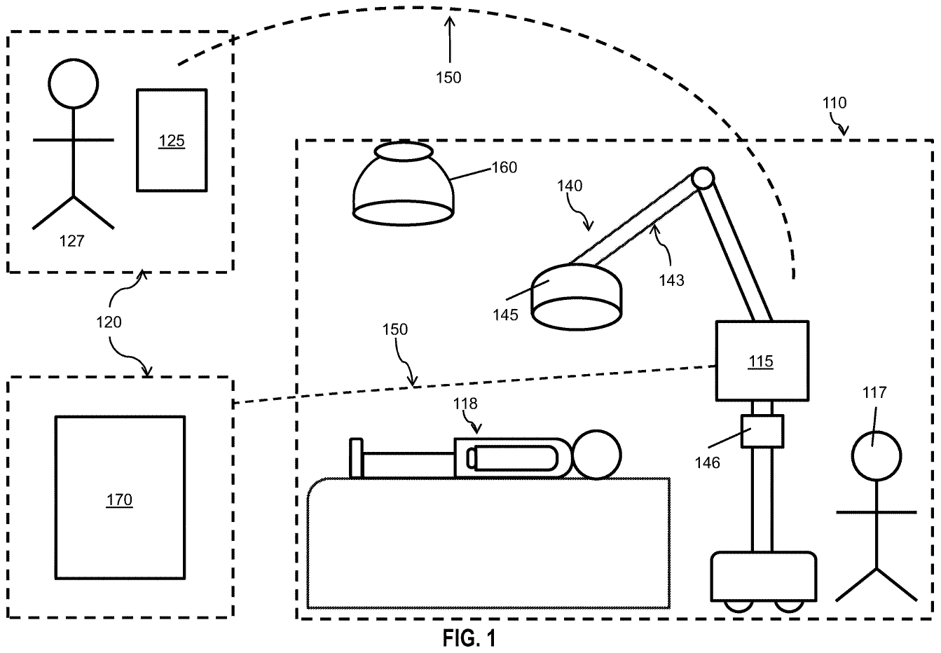

FIG. 1 show examples of an audio capture system that may be utilized within a medical suite to monitor, capture, and enhance audio communications. The audio capture system may comprise the one or more audio recording devices as described above. In some alternative embodiments, the audio capture system may comprise one or more imaging devices. In some cases, the audio recording devices may be integrated with the one or more imaging devices. In other cases, the audio recording devices may be separate and distinct from the one or more imaging devices. The audio capture system may be configured to capture audio communications relating to a surgical procedure, or audio communications made at or near a surgical site or an operating environment in which a surgical procedure is being performed.

The audio capture system may be configured to capture audio communications made in a first location 110. In some cases, the audio communications captured at the first location 110 may be processed and/or enhanced using an audio enhancement module that is located in the first location 110. In other cases, the audio communications captured at the first location 110 may be transmitted to a second location 120 for processing and/or enhancement. In some cases, the first location 110 and the second location 120 may be in a same operating room or healthcare facility. In other cases, the first location 110 may be in an operating room or a healthcare facility, and the second location 120 may be a location remote from the operating room or healthcare facility. In some cases, the audio capture system may also comprise a local communication device 115. In some cases, the local communication device 115 may be operably coupled to the one or more audio recording devices described above. The local communication device 115 may optionally communicate with a remote communication device 125 (e.g., a mobile device of a remote user 127), or a remote sever 170. In some cases, the remote server 170 may be configured to process and/or enhance audio communications recorded at the first location 110.

In some embodiments, audio communications from the first location 110 may be transmitted to a second location 120 using a local communication device 115 that is configured to communicate with a remote communication device 125 via a communication channel 150. Any types of communication channel 150 may be formed between the remote communication device and the local communication device. The communication channel may be a direct communication channel or an indirect communication channel. The communication channel may employ wired communications, wireless communications, or both. The communications may occur over a network, such as a local area network (LAN), wide area network (WAN) such as the Internet, or any form of telecommunications network (e.g., cellular service network). Communications employed may include, but are not limited to 3G, 4G, LTE communications, and/or Bluetooth, infrared, radio, or other communications. Communications may optionally be aided by routers, satellites, towers, and/or wires. The communications may or may not utilize existing communication networks at the first location and/or second location.

The first location 110 may be a medical suite, such as an operating room of a health care facility. A medical suite may be within a clinic room or any other portion of a health care facility. A health care facility may be any type of facility or organization that may provide some level of health care or assistance. In some examples, health care facilities may include hospitals, clinics, urgent care facilities, out-patient facilities, ambulatory surgical centers, nursing homes, hospice care, home care, rehabilitation centers, laboratory, imaging center, veterinary clinics, or any other types of facility that may provide care or assistance. A health care facility may or may not be provided primarily for short term care, or for long-term care. A health care facility may be open at all days and times, or may have limited hours during which it is open. A health care facility may or may not include specialized equipment to help deliver care. Care may be provided to individuals with chronic or acute conditions. A health care facility may employ the use of one or more health care providers (a.k.a. medical personnel/medical practitioner). Any description herein of a health care facility may refer to a hospital or any other type of health care facility, and vice versa.

The first location 110 may be any room or region within a health care facility. For example, the first location may be an operating room, surgical suite, clinic room, triage center, emergency room, or any other location. The first location may be within a region of a room or an entirety of a room. The first location may be any location where an operation may occur, where surgery may take place, where a medical procedure may occur, and/or where a medical product is used. In one example, the first location may be an operating room with a patient 118 that is being operated on, and one or more medical personnel 117, such as a surgeon or surgical assistant that is performing the operation, or aiding in performing the operation. Medical personnel may include any individuals who are performing the medical procedure or aiding in performing the medical procedure. Medical personnel may include individuals who provide support for the medical procedure. For example, the medical personnel may include a surgeon performing a surgery, a nurse, an anesthesiologist, and so forth. Examples of medical personnel may include physicians (e.g., surgeons, anesthesiologists, radiologists, internists, residents, oncologists, hematologists, cardiologists, etc.), nurses (e.g., CNRA, operating room nurse, circulating nurse), physicians' assistants, surgical techs, and so forth. Medical personnel may include individuals who are present for the medical procedure and authorized to be present.

In some cases, the second location 120 may be in a same operating room or healthcare facility as the first location 110. In other cases, the second location 120 may be any location that is remote from the first location 110. For instance, if the first location is a hospital, the second location may be outside the hospital. In some instances, the first and second locations may be within the same building but in different rooms, floors, or wings.

In some embodiments, one or more audio recording devices may be provided at or near the first location 110. The one or more audio recording devices may or may not be supported by a medical console 140. In some embodiments, the one or more audio recording devices may be supported by a ceiling 160, wall, furniture, or other items at the first location. For instance, one or more audio recording devices may be mounted on a wall, ceiling, or other device. Such audio recording devices may be directly mounted to a surface, or may be mounted on a boom or arm. For instance, an arm may extend down from a ceiling while supporting an audio recording device. In another example, an arm may be attached to a patient's bed or surface while supporting an audio recording device. In some instances, an audio recording device may be worn by medical personnel. For instance, an audio recording device may be worn on a headband, wrist-band, torso, or any other portion of the medical personnel. An audio recording device may be part of a medical device or may be supported by a medical device (e.g., endoscope, etc.). The one or more audio recording devices may be fixed or movable. The one or more audio recording devices may be capable of rotating about one or more, two or more, or three or more axes. The one or more audio recording devices may be adjusted using pan-tilt-zoom operations. The audio recording devices may be manually moved by an individual at the first location. The audio recording devices may be locked into position and/or unlocked to be moved. In some instances, the one or more audio recording devices may be remotely controlled by one or more remote users. The position and/or orientation of the audio recording devices may be adjusted to modify a detection range or a detection area associated with the audio recording devices.

In some cases, the one or more audio recording devices may be provided on a medical console 140. The medical console 140 may optionally include one or more audio recording devices 145, 146. In other cases, the one or more audio recording devices may be positioned on a distal end of an articulating arm 143 of the medical console 140. The audio communications captured by the one or more audio recording devices 145, 146 may be processed and enhanced using an audio processing module. The audio communications may be processed and enhanced in real-time as they are captured. The audio communications may be sent to a remote communication device that is configured to remotely receive the audio communications and provide the audio communications to an audio enhancement module that is configured to enhance the audio communications captured by the audio recording devices.

In some cases, enhancing the audio communications may occur locally at the first location 110. In some embodiments, the enhancement may occur on-board a medical console 140. For instance, the enhancement may occur with aid of one or more processors of a communication device 115 or another computer that may be located at the medical console. In some instances, the enhancement may occur remotely from the first location. In some instances, one or more servers 170 may be utilized to perform audio analysis and enhancement. The server may be able to access and/or receive information from multiple locations and may collect one or more datasets. The datasets may be used in conjunction with machine learning in order to provide increasingly accurate audio analysis and/or enhancement. Any description herein of a server may also apply to any type of cloud computing infrastructure. The analysis may occur remotely, and feedback may be communicated back to the console and/or location communication device in substantially real-time. Any description herein of real-time may include any action that may occur within a short span of time (e.g., within less than or equal to about 10 minutes, 5 minutes, 3 minutes, 2 minutes, 1 minute, 30 seconds, 20 seconds, 15 seconds, 10 seconds, 5 seconds, 3 seconds, 2 seconds, 1 second, 0.5 seconds, 0.1 seconds, 0.05 seconds, 0.01 seconds, or less).

In some embodiments, the communication devices 115, 125 may comprise one or more microphones or speakers. A microphone may comprise an audio detection device that is configured to capture audible sounds such as the voice of a user or the speech of medical personnel in the first location. One or more speakers may be provided to play sound (e.g., the audio communications or the enhanced audio communications). For instance, a speaker on a remote communication device 125 may allow an end user in the second location to hear sounds captured by a local communication device 115 in the first location, and vice versa. In some embodiments, an audio enhancement module may be provided. The audio enhancement module may be supported by a video capture system for monitoring surgical procedures. The audio enhancement module may comprise an array of microphones that may be configured to clearly capture voices within a noisy room while minimizing or reducing background noise or audio communications by other persons or objects with a lower priority. The audio enhancement module may be separable or may be integral to the video capture system.

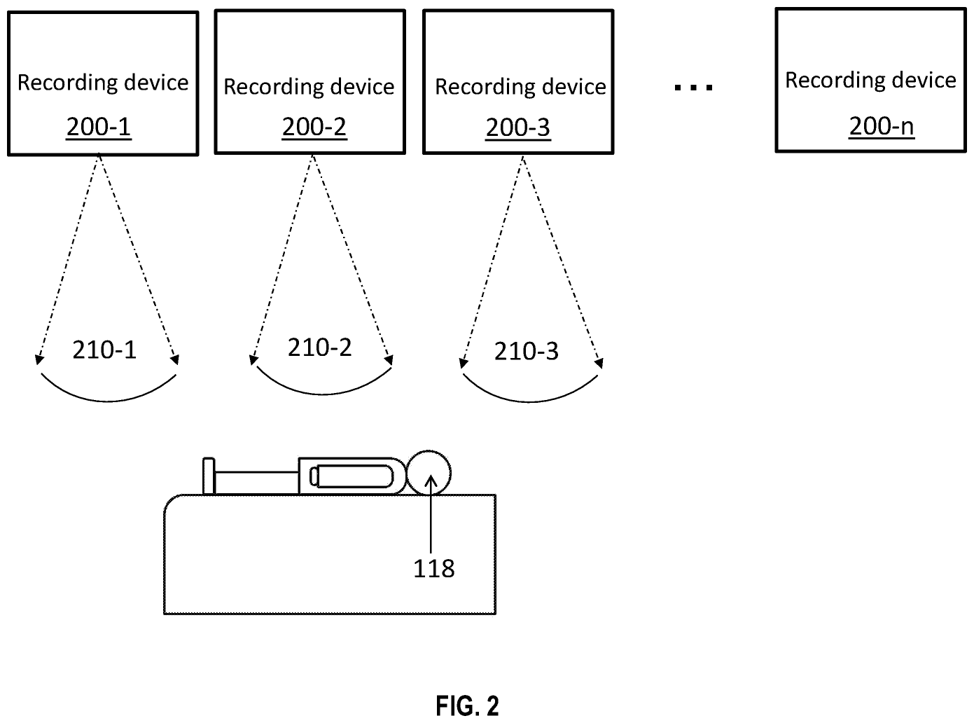

FIG. 2 illustrates a plurality of audio recording devices comprising one or more audio recording devices 200-1, 200-2, and 200-3. The one or more audio recording devices may be provided in a medical suite where a surgical operation may be performed on a medical patient 118. The plurality of audio recording devices 200-n may comprise n number of audio recording devices, where n is greater than or equal to 1. Each of the recording devices may have a corresponding detection range or detection area 210-1, 210-2, and 210-3 associated with the recording devices. The detection ranges or detection areas 210-1, 210-2, and 210-3 may be focused or oriented in a particular direction relative to the recording devices (herein referred to as directionality or directivity). Each of the detection areas may correspond to an area or a range in which the recording device may register, record, and/or capture audio communications above a certain threshold volume. The detection areas for the audio recording devices may overlap or partially overlap. In some cases, the detection areas for the audio recording devices may be different and/or may not overlap. In some cases, the detection areas may be adjusted or modified by changing a position and/or an orientation of the audio recording devices. In other cases, the detection areas may be adjusted or modified using beam forming and/or beam steering.

The present disclosure provides systems and methods for enhancing audio communications. In some cases, enhancing audio communications may comprise improving a transmission or reception quality of an audio communication, increasing a signal to noise ratio for one or more portions of an audio communication, and/or augmenting an audio communication with additional data or information. In other cases, enhancing audio communications may comprise prioritizing one or more portions of an audio communication relative to other portions of the audio communication, or prioritizing one or more audio communications relative to a plurality of audio communications. In some cases, enhancing audio communications may comprise adjusting a detection range, a detection area, a directionality, and/or a directivity of one or more audio detection devices, based on a content of an audio communication or an identity of a source of an audio communication. In some cases, enhancing audio communications may comprise adjusting a sensitivity of one or more audio detection devices to audio communications received from a certain area or region, or from a certain speaker or source.

As used herein, an audio communication may refer to any communication that is based on sound or speech. In some cases, the audio communication may comprise one or more acoustic waveforms or signals corresponding to speech and/or one or more sounds generated by a human, an animal, a machine (e.g., medical equipment), a physical object, natural phenomena, and/or any physical, biological, or chemical interaction or reaction that creates acoustic waveforms that may propagate through a transmission medium. The transmission medium may comprise a gas, a liquid, or a solid. The audio communication may be captured or recorded using one or more microphones or microphone arrays. The one or more microphones may capture audible sounds such as the voice of a person who is within a detection range of the one or more microphones.

The systems and methods of the present disclosure may be used to enhance audio communications in real-time as audio communications are being received or transmitted. In some cases, the systems and methods of the present disclosure may be used to enhance audio quality by processing one or more audio communications and generating enhanced audio communications within a predetermined time after an audio communication is received or transmitted.

In one aspect, the present disclosure provides a method for enhancing audio communications. The method may comprise (a) detecting one or more parameters associated with a medical procedure and one or more audio communications associated with the medical procedure; and (b) processing the one or more audio communications based on the one or more parameters to generate one or more enhanced audio communications.

In some embodiments, the one or more parameters may comprise a physical feature, a face, a voice, or an identity of a human or a robot that made the one or more audio communications. In some embodiments, the one or more parameters may comprise a key word, phrase, or sentence of the one or more audio communications.

In some embodiments, processing the one or more audio communications may comprise beam forming to adjust a detection area, a detection range, a directivity, or a directionality of one or more audio detection devices. In some embodiments, processing the one or more audio communications may comprise prioritizing a detection or a capture of the one or more audio communications based on an identity of a speaker. In some embodiments, processing the one or more audio communications may comprise adjusting the priority of detection or capture based on a detection of one or more key words, phrases, or sentences in the one or more audio communications.

In some cases, the systems and methods of the present disclosure may be used to enhance audio communications using one or more control voltage (CV) signals. The one or more CV signals may comprise an analog or digital signal. In some cases, the one or more CV signals may be used to adjust one or more audio characteristics of an audio communication. The one or more audio characteristics may comprise, for example, a frequency of the audio communication, a wavelength of the audio communication, an amplitude of the audio communication, a pitch associated with the audio communication, a tone associated with the audio communication, and/or an intensity or loudness associated with the audio communication.

In some cases, the systems and methods of the present disclosure may be used to enhance audio quality using natural language processing (NLP). NLP may comprise manipulating and/or processing natural language such as speech and text in order to derive information or data associated with the speech and/or text (e.g., information about upcoming critical steps in a surgical procedure, a certain type of tool needed to complete a surgical step, or a specific type of support needed for a particular surgical step).

In some cases, the system and methods of the present disclosure may be used to enhance audio quality using speaker recognition. Speaker recognition may comprise identifying a speaker or a source of an audio communication based on one or more characteristics of the audio communication. The one or more characteristics may comprise, for example, a frequency of the audio communication, a wavelength of the audio communication, and/or an amplitude of the audio communication. In some cases, the one or more characteristics may comprise a pitch associated with the audio communication, a tone associated with the audio communication, and/or an intensity or loudness associated with the audio communication.

In some cases, the system and methods of the present disclosure may be used to enhance audio quality based on face detection. Face detection may comprise detecting or identifying a person based on one or more images or videos of a facial feature of the person. The facial feature may comprise a physical feature of one or more portions of a person's face (e.g., an eye, a nose, an ear, a mouth, hair, a facial structure, etc.). The one or more images or videos of a facial feature of the person may be obtained using an imaging device (e.g., a camera, a video camera, an imaging sensor, etc.) In some cases, face detection may comprise identifying a location of a person based on one or more images or videos of the person. In some cases, face detection may comprise associating a person with a certain location or area that is within a detection range of an imaging device.

In some cases, the system and methods of the present disclosure may be used to enhance audio quality based on a detection of other identifying features associated with a person (e.g., a body part other than the face, such as a hand of a person). In some cases, the other identifying features may comprise, for example, a tone, a rhythm, and/or a cadence of a person's speech, or a particular mannerism associated with a person (e.g., a gait or any other repeated or habitual movement).

In some cases, audio enhancement may be implemented using real-time beam forming. Beamforming (or spatial filtering) may refer to a signal processing technique used in sensor arrays (e.g., a microphone array) for directional signal transmission or reception. Beamforming may be used to enhance signals from a desired direction relative to a microphone array and to suppress noise and interferences from other directions. Beamforming may be achieved by combining elements in an antenna array in such a way that signals at particular angles experience constructive interference while others experience destructive interference. Beamforming can be used at both the transmitting and receiving ends in order to achieve spatial selectivity. Beamforming may be used to enhance the detection of audio communications from a particular source, based on an identity of the source or the contents of the communication made by the source.

In some cases, beamforming may be used to extract sound sources in a room and distinguish between multiple speakers in the room. Beamforming may be implemented based on a prior or current location of a speaker, which may be known in advance or determined based on face detection. In some cases, the location of a speaker may be determined based on a time of arrival for an audio communication transmitted from an audio source to one or more microphones.

Beam forming may be used to improve detection of audio signals that are received within a predetermined detection range corresponding to a directionality or directivity of one or more microphones. In some embodiments, the predetermined detection area may be about +/−60° from a center point corresponding to a position or a location of a primary doctor. In other embodiments, the predetermined detection area may be about +/−10° from a center point corresponding to a position or a location of one or more parties of interest. In some cases, the systems and methods of the present disclosure may be implemented based on a priority list comprising the one or more parties of interest. The priority list may comprise a list of individuals who are supporting and/or performing a surgical operation. Individuals with a higher priority may have their audio communications prioritized and captured over the audio communications of individuals with a lower priority.

In general, at any given point of time, the systems and methods of the present disclosure may be used to generate “N” number of beams with a detection area of “+/−X°” relative to one or more points of interest. The one or more points of interest may correspond to a position or a location of an object or a person of interest. In some cases, the detection area may range from about +/−1° to about +/−90° relative to one or more points of interest.

Prior to a surgical procedure, one or more profiles can be set up for doctors, surgeons, assistants, or other medical staff. Various priorities may be assigned for each individual, either automatically or based on a predetermined preference. The systems and methods of the present disclosure may be implemented to create N number of beams with a detection area of “+/−X°” relative to one or more points or persons of interest. In some cases, the detection area may range from about +/−1° to about +/−90° relative to one or more points or persons of interest.

In some embodiments, one or more microphones (or any other audio recording or audio detection devices) may be configured to recognize and/or identify one or more speakers based on (i) audio communications presently made by the one or more speakers and (ii) one or more historical records of prior audio communications made by the one or more speakers. The one or more microphones may be configured to prioritize detection of audio communications made by one or more persons of interest based on the recognition of the persons of interest and a priority level assigned to the persons of interest. In some embodiments, the one or more microphones may be configured to recognize and/or identify one or more tools or products used in surgery based on audio communications made by one or more speakers. For example, the microphones may be used to detect key words spoken by a doctor, medical worker, or support staff, and to identify a tool or product referenced by the doctor, medical worker, or support staff through the key words. In some cases, the doctor, medical worker, or support staff may request a particular tool or product to aid in the performance of one or more tasks or steps associated with a procedure, and the one or more microphones may detect that the tool or product has been requested. Upon detecting that a particular tool or product has been requested, the systems disclosed herein may transmit a notification or a request to one or more individuals or entities assisting with the procedure to retrieve or access the tool or product requested by the doctor or surgeon.

In some embodiments, natural language processing (NLP) may be used to interpret and process audio communications made by a doctor or a surgeon before and/or during a procedure. The NLP may be performed using one or more algorithms. In some cases, the NLP may comprise context aware NLP that can interpret audio communications to understand, determine, or identify (i) what kind of surgery is being performed and/or (ii) which tools and/or products are being used. In some embodiments, the context aware NLP may also be used to catalog (i) different steps in the procedure and/or (ii) the tools or products used by a doctor or a hospital for surgical or medical procedures. In some cases, NLP may be used to generate or compile data (e.g., statistics) on the timing of steps in a surgical procedure or the volume or frequency of usage for various tools, products, or medical instruments. In some cases, NLP may be used to determine, for instance, success rates and/or failure rates for different procedures or procedural steps that are identified using NLP. In other cases, NLP may be used to determine success rates and/or failure rates for different procedures that are performed using particular tools or products that are identified by way of NLP.

In some cases, the one or more microphones may be configured to detect a voice of a person of interest and/or voice activity of a person of interest, and to prioritize detection of audio communications made by the person of interest based on (i) the detection of the voice or voice activity of the person of interest and (ii) a priority level assigned to the person of interest. For example, when the one or more microphones do not detect a voice or voice activity of a person of interest, the one or more microphones may not or need not prioritize any audio communications made by multiple parties. However, when the one or more microphones detect a voice or voice activity of a person of interest, the one or more microphones may prioritize the audio communications made by the person of interest over other audio communications made by other persons or persons of interest with a lower assigned priority.

In some cases, the systems and methods of the present disclosure may be implemented to adjust the beamforming capabilities described herein based on a detected location or position of one or more persons of interest. For example, if the directionality or directivity of one or more microphones corresponds to a first detection range or area and the location or position of one or more persons of interest requires adjustment of the directionality or directivity to a second detection range or area, the directionality or directivity of the one or more microphones may be modified or adjusted to correspond to the second detection range or area. The first detection range or area the second detection range or area may overlap or partially overlap. In some cases, the first detection range or area the second detection range or area may be different. Adjusting the directionality or directivity of the one or more microphones may comprise one or more aspects of beam steering.

In some cases, the systems and methods of the present disclosure may be implemented to facilitate speech detection. Speech detection may comprise detecting a presence or an absence of speech or other audio communications, or identifying a speaker based on one or more audio communications received by an audio recording device (e.g., a microphone or an array of microphones). In some cases, speech detection may comprise detecting or identifying important key words or sentences spoken by medical operators, doctors, surgeons, medical staff, and/or any persons of interest. In some cases, such speech detection may be used to change or adjust a priority of one or more individuals, based at least in part on the important key words, phrases, or sentences spoken by the one or more individuals.

In some cases, the priority of one or more individuals may be adjusted based on certain words, phrases, or sentences spoken by the one or more individuals. As described above, the priorities assigned to individuals may be used to prioritize detection of audio communications made by those individuals over other persons who may be nearby. In some cases, the one or more individuals may comprise at least one person who is listed on a priority list. In other cases, the one or more individuals may comprise at least one person who is not listed on a priority list. In such cases, when an individual not on a priority list makes a statement comprising one or more important key words, phrases, or sentences, such individual may be added to the priority list. Further, the priority of other individuals on the priority list may be adjusted to accommodate the addition of another individual to the priority list.

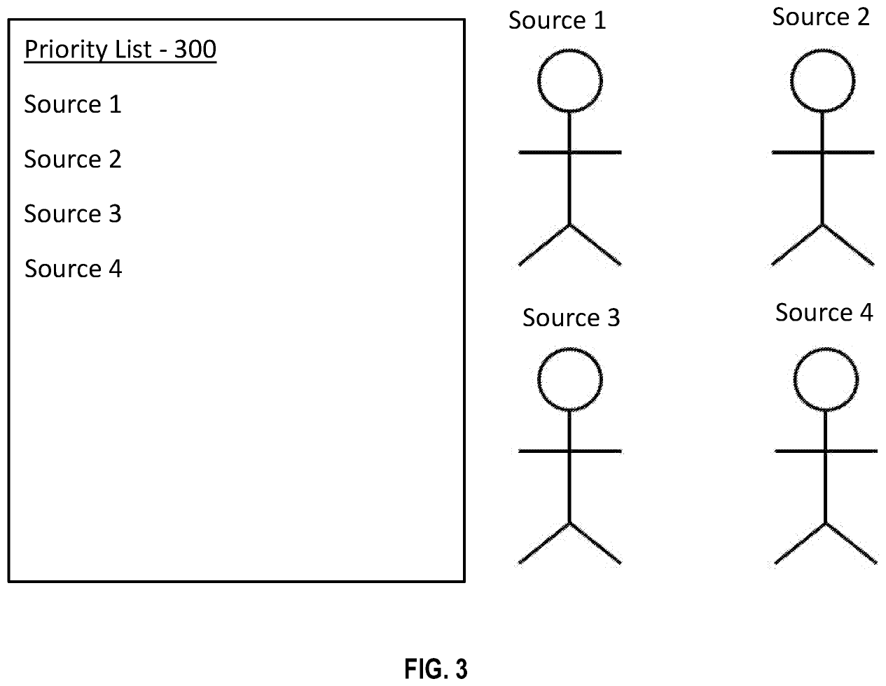

FIG. 3 illustrates an example of a priority list 300 that may be used to prioritize detection of audio communications. In one example, a plurality of individuals may be present in an operating room. The plurality of individuals may be treated as a plurality of audio sources (e.g., source 1, source 2, source 3, and source 4). The priority list 300 may assign a priority to each audio source such that the audio recording devices described herein would prioritize detection of audio communications from those audio sources with a higher priority. For example, if the priority list designates source 1 with the highest priority, source 2 with the second highest priority, source 3 with the third highest priority, and source 4 with the lowest priority, one or more of the audio detection devices may be configured to prioritize audio communications from source 1 over the audio communications from source 2, source 3, and/or source 4.

In some cases, the priority list may be adjusted based on the content of the speech. For example, if source 2 communicates one or more key words, phrases, or sentences, then source 2 may be prioritized over source 1 for at least a predetermined period of time. In other cases, the priority list may be adjusted to include another source (e.g., a source 5) when another individual makes an audible communication that requires prioritization over other audio sources.

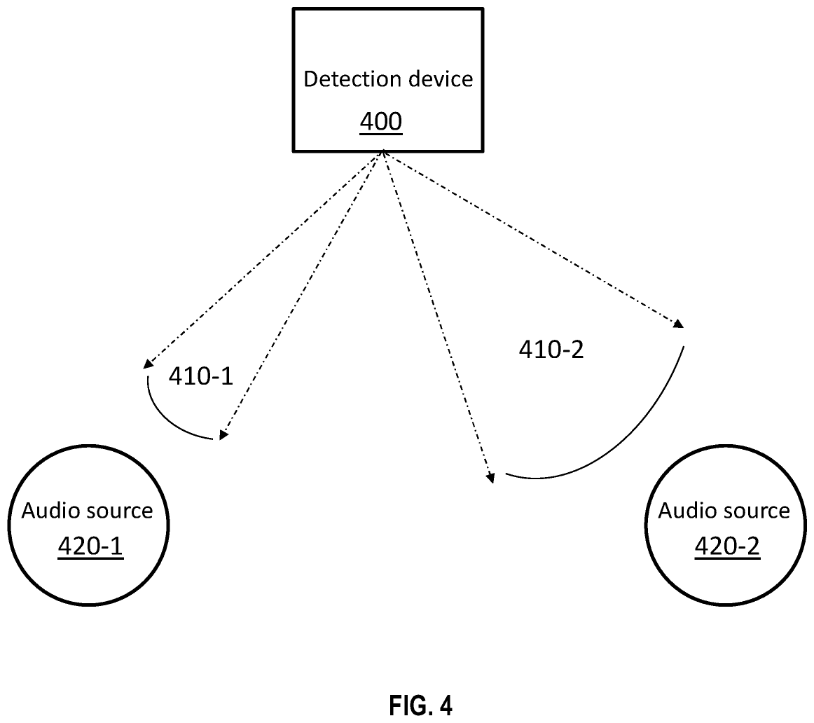

FIG. 4 illustrates one or more beams 410-1, 410-2 that may be generated for an audio detection device. As used herein, an audio detection device may be referred to interchangeably as an audio recording device. The audio detection device may comprise, for example, one or more microphones or microphone arrays for detecting, recording, and/or receiving audio communications. The one or more beams 410-1, 410-2 may correspond to different detection areas and/or different detection ranges. In some cases, the orientation and/or the angular coverage of the one or more beams 410-1, 410-2 may be adjusted to prioritize one or more audio communications among a plurality of audio communications made by a plurality of audio sources 420-1, 420-2. Such prioritization may be in response to, for example, a priority list or a change to the priority list; a recognition of certain key words, phrases, or sentences; and/or an identification of a particular voice or speech made by a particular individual.

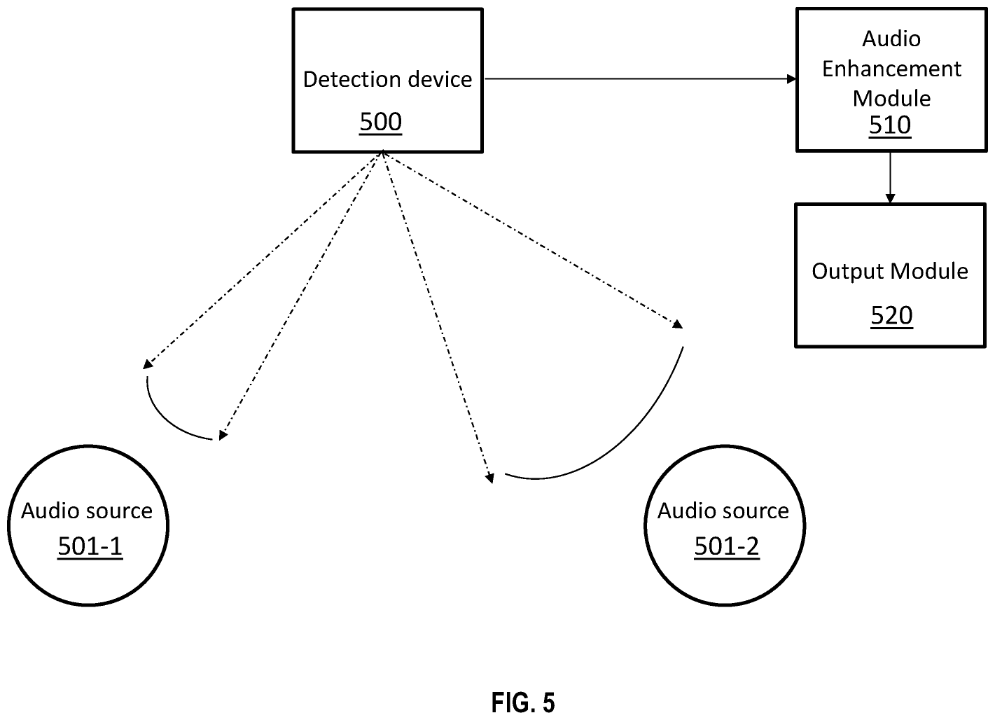

FIG. 5 illustrates an exemplary system for detecting and enhancing audio communications. The system may comprise an audio detection device 500 that is configured to detect audio communications originating from one or more audio sources 501-1, 501-2. The audio detection device 500 may be configured to receive audio communications and to transmit the audio communications to an audio enhancement module 510 that is configured to enhance the audio communications using any of the audio enhancement methods described herein. The audio enhancement module 510 may be further configured to transmit the enhanced audio communications to an output module or device 520, such as a speaker. In some cases, the speaker may be integrated into a computing device located within an operating room or a healthcare facility. In other cases, the speaker may be integrated into a computing device that is remote from the operating room or healthcare facility. In some cases, the enhanced audio communications may be provided to an individual located in the operating room or the healthcare facility. In other cases, the enhanced audio communications may be provided to a medical device or a robot that is configured to use the enhanced audio communications to aid a surgical procedure or a surgical operator who is performing a surgical procedure.

In any of the embodiments described herein, machine learning may be used to train the audio enhancement systems of the present disclosure to improve a detection of audio communications with a high priority. In some cases, one or more data sets corresponding to high priority audio communications may be provided to a machine learning module. The machine learning module may be configured to generate machine learning data based on the data sets. The one or more data sets may be used as training data sets for one or more machine learning algorithms. Learning data may be generated based on the data sets. In some embodiments, supervised learning algorithms may be used. Optionally, unsupervised learning techniques and/or semi-supervised learning techniques may be utilized in order to generate learning data. The learning data may be useful for detecting and/or recognizing high priority audio communications. The learning data may be used to train the machine learning module and/or the machine learning algorithms to detect and/or recognize high priority audio communications. In some cases, data associated with one or more high priority audio communications detected by the audio enhancement system using a machine learning algorithm may be fed back into the learning data sets to improve the machine learning algorithms.

In some embodiments, the machine learning module may utilize one or more neural networks. The one or more neural networks may comprise, for example, a deep convolution neural network. The machine learning may utilize any type of convolutional neural network (CNN). Shift invariant or space invariant neural networks (SIANN) may also be utilized. Image classification, object detection, and/or object localization may also be utilized. In some embodiments, the neural network may comprise a convolutional neural network (CNN). The CNN may be, for example, U-Net, ImageNet, LeNet-5, AlexNet, ZFNet, GoogleNet, VGGNet, ResNet18, or ResNet, etc. In some cases, the neural network may be, for example, a deep feed forward neural network, a recurrent neural network (RNN), LSTM (Long Short Term Memory), GRU (Gated Recurrent Unit), Auto Encoder, variational autoencoder, adversarial autoencoder, denoising auto encoder, sparse auto encoder, Boltzmann machine, RBM (Restricted BM), deep belief network, generative adversarial network (GAN), deep residual network, capsule network, attention/transformer networks, etc. In some embodiments, the neural network may comprise one or more neural network layers. The neural network may have at least about 2 to 1000 or more neural network layers. In some cases, the machine learning algorithm may implement, for example, a random forest, a boosted decision tree, a classification tree, a regression tree, a bagging tree, a neural network, or a rotation forest.

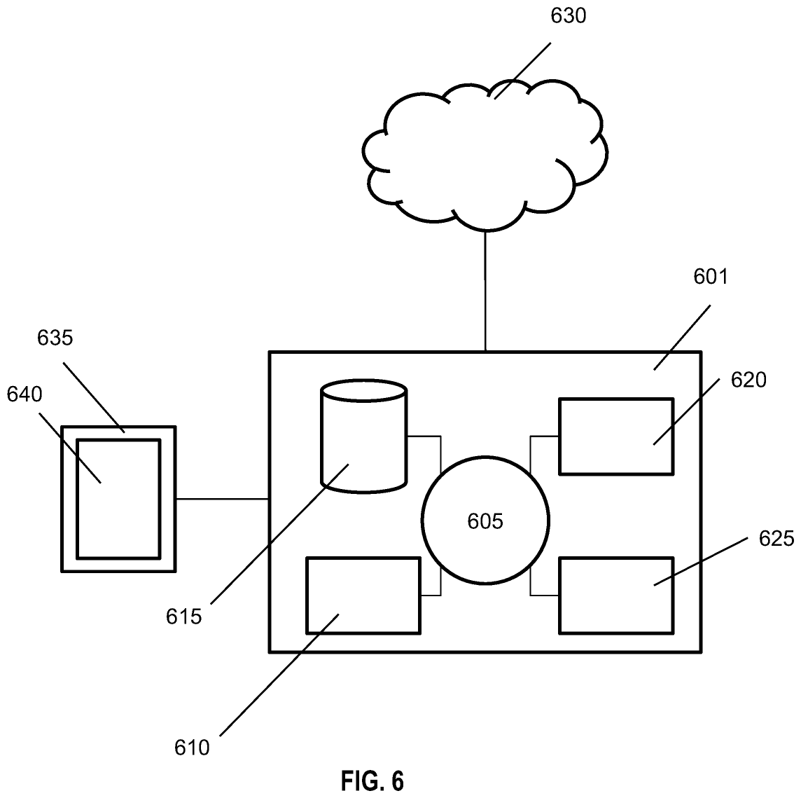

In an aspect, the present disclosure provides computer systems that are programmed or otherwise configured to implement methods of the disclosure, e.g., any of the subject methods for enhancing audio communications. FIG. 6 shows a computer system 601 that is programmed or otherwise configured to implement a method for enhancing audio communications. The computer system 601 may be configured to, for example, (a) detect one or more parameters associated with a medical procedure and one or more audio communications associated with the medical procedure; and (b) process the one or more audio communications based on the one or more parameters to generate one or more enhanced audio communications. The computer system 601 can be an electronic device of a user or a computer system that is remotely located with respect to the electronic device. The electronic device can be a mobile electronic device.

The computer system 601 may include a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are located external to the computer system 601 (e.g., on a remote server that is in communication with the computer system 601 through an intranet or the Internet).

The computer system 601 can communicate with one or more remote computer systems through the network 630. For instance, the computer system 601 can communicate with a remote computer system of a user (e.g., a medical operator, a medical assistant, or a remote viewer monitoring the medical operation). Examples of remote computer systems include personal computers (e.g., portable PC), slate or tablet PC's (e.g., Apple® iPad, Samsung® Galati Tab), telephones, Smart phones (e.g., Apple® iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, a portal for a medical worker to (i) monitor a detection of one or more audio communications made during a medical procedure and (ii) receive one or more enhanced audio communications from an audio enhancement module that is configured to process the one or more audio communications. The portal may be provided through an application programming interface (API). A user or entity can also interact with various elements in the portal via the UI. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 605. For example, the algorithm may be configured to (a) detect one or more parameters associated with a medical procedure and one or more audio communications associated with the medical procedure; and (b) process the one or more audio communications based on the one or more parameters to generate one or more enhanced audio communications.

In another aspect, the present disclosure provides systems and methods for audio beam selection. One or more individuals viewing a live stream of a surgical procedure or a recording of a surgical procedure may select one or more audio beams or audio channels of interest from a plurality of different audio beams or audio channels. The audio beams or audio channels of interest may correspond to different individuals supporting or viewing the surgical procedure (e.g., different specialists, doctors, or remote vendor representative). In some cases, the audio beams or audio channels of interest may correspond to a usage or an operation of various different surgical tools or instruments. In some cases, the plurality of audio beams or audio channels may be associated with a plurality of different cameras capturing different views or different phases of an ongoing surgical procedure.

In some cases, multiple cameras may be connected or operatively coupled to a medical console located in a healthcare facility. The multiple cameras may be configured to provide multiple views of an ongoing surgical procedure. The multiple cameras may each have one or more audio recording or detection devices (e.g., microphones) to augment the images or videos captured using the multiple cameras. The multiple cameras may be used to capture images or videos of the surgical scene, and the images or videos along with any associated audio may be provided to one or more individuals through a live stream or in the form of a video recording. Such video recording may be stored in a library or a server (e.g., a cloud server) so that the one or more individuals can access the video at any time after the video is recorded.

In some cases, one or more individuals may simultaneously mark a phase of a surgical procedure and select or extract audio associated with the phase of the surgical procedure. This may allow the individuals to hear only a portion of the audio associated with a video of a surgical procedure. The individuals may each select different phases of interest, and listen to different audio clips associated with different phases of the surgical procedure. In some cases, the individuals may select a same phase of interest, and listen to different audio clips associated with different views of the surgical procedure, a usage or operation of different surgical instruments, and/or different speakers who are assisting with the surgical procedure or providing audio commentary pertaining to the performance of the surgical procedure.

In some cases, an individual may only be concerned with audio communications associated with a particular instrument, a particular specialist, or a particular doctor. The systems and methods of the present disclosure may permit a first individual to listen to audio communications by a first speaker, and a second individual to listen to audio communications by a second speaker. In some cases, a first individual may listen to audio communications associated with a first instrument or a first doctor or specialist, and a second individual to listen to audio communications associated with a second instrument or a second doctor or specialist. The first individual and/or the second individual may be, for example, a remote specialist, a vendor representative, a doctor, a surgeon, a surgical assistant, a medical worker, a medical resident, a medical intern, a medical student, or any other individual who is interested in viewing the surgical procedure and/or listening to audio communications associated with the surgical procedure (e.g., a friend or a family member of the subject who is undergoing the surgical procedure). The first speaker and/or the second speaker may be, for example, a remote specialist, a vendor representative, a doctor, a surgeon, a surgical assistant, or a medical worker.

In some cases, multiple individuals may select audio beams or channels of interest by selecting a desired audio beam or channel from a master list of audio devices or audio channels. The master list of audio devices or audio channels may be generated for each surgical procedure. The list may be compiled manually, or automatically generated based on a detection of one or more audio recording devices that are being used to record audio communications during a surgical procedure.

In other cases, multiple individuals may select audio beams or channels of interest by selecting an instrument, a specialist, doctor, surgeon, or surgical phase of interest. In such cases, post-processing of a surgical video may be performed to extract the associated audio beams or channels. For example, a first individual may view a surgical video and select a particular instrument, specialist, doctor, surgeon, or surgical phase of interest. One or more processors may be used to post-process the surgical video to extract the relevant audio communications associated with the particular instrument, specialist, doctor, surgeon, or surgical phase of interest selected by the first individual. In parallel, a second individual may view the same surgical video and select a particular instrument, specialist, doctor, surgeon, or surgical phase of interest. One or more processors may be used to post-process the surgical video to extract the relevant audio communications associated with the particular instrument, specialist, doctor, surgeon, or surgical phase of interest selected by the second individual.

As used herein, post-processing may comprise receiving audio from multiple channels and determining or extracting a particular audio stream or channel of interest based on a selection or input provided by an individual. The selection or input may be with respect to a particular instrument, specialist, doctor, surgeon, or surgical phase of interest. The selection or input may comprise a physical input (e.g., clicking on a particular speaker or a particular instrument within a surgical video).

In some cases, metadata may be tracked to extract one or more audio streams of interest from multiple streams. The metadata may comprise information associating the one or more audio streams of interest with a particular instrument, specialist, doctor, surgeon, or surgical phase of interest. The metadata may be generated based on an identification or detection of various instruments, specialists, doctors, surgeons, or surgical phases of interest using, for example, computer visions techniques or one or more machine learning or classification algorithms.

In some cases, once a particular audio channel or audio stream of interest is identified and selected, the systems and methods of the present disclosure may be used to amplify the audio channel or audio stream of interest. Further, the systems and methods of the present disclosure may be used to attenuate other audio channels or audio streams that are not of interest. The level of amplification or attenuation may be adjusted based on, for example, a user preference or an input provided by a user.

In some cases, one or more users may be automatically assigned to one or more particular audio streams or channels from a plurality of audio streams or channels. The users may be assigned to a particular set of audio streams or channels based on, for example, an identity or a role of the users. In some instances, a first user (e.g., a product support specialist) may be automatically assigned to a first audio stream or channel and a second user (e.g., a consulting doctor) may be automatically assigned to a second audio stream or channel. The first audio stream or channel may comprise audio communications associated with one or more products (e.g., tools, instruments, devices, or systems) that the product support specialist is familiar with and/or knowledgeable of. In some cases, the first audio stream or channel may comprise audio communications associated with a usage of one or more products that the product support specialist is familiar with and/or knowledgeable of. The first audio stream or channel may comprise audio communications that provide the product support specialist with information on the identity or the usage of the one or more products so that the product support specialist can provide specialized guidance for how to prepare or use the one or more products properly or effectively. The second audio stream or channel may comprise, for example, audio communications associated with another aspect of a surgical procedure (e.g., audio communications associated with the performance of one or more steps of the surgical procedure, or procedural aspects of the surgical procedure including medical or surgical techniques). The second audio stream or channel may comprise audio communications that provide the consulting doctor with information on how a surgeon is performing a procedure so that the consulting doctor can provide specialized guidance for how to perform one or more steps of the surgical procedure properly or more effectively. In some cases, the first and second audio streams or channels may comprise a same or similar audio content. In other cases, the first and second audio streams or channels may comprise different audio content. The different audio content may comprise audio communications made by different individuals or audio communications associated with different aspects or portions of a surgical procedure.

In some cases, one or more audio streams may be automatically filtered from a plurality of audio streams and presented to a particular user or a particular subset of users based on the identity of the users, the role of the users, or the content of the audio streams. In other cases, the filtering and assignment of the one or more audio streams to a particular user or subset of users may be adjusted or modified. For example, if one or more users want to listen to various audio streams or channels that are not automatically assigned to them, the one or more users may provide one or more inputs to change or add other audio streams or channels of interest. In some cases, users may also provide an input to change or remove audio streams or channels that are no longer of interest. The inputs may comprise, for example, a manual selection or removal of one or more audio streams. In some cases, such manual selection or removal of audio streams may be made with respect to or with reference to a master list of audio streams or channels. In some cases, the inputs may be analyzed and used to change one or more parameters or factors used to make the initial automatic assignment of audio channels or streams to the users. In some cases, the selection or assignment of audio channels or streams may be varied directly by a particular user. In other cases, the selection or assignment of audio channels or streams may be varied by a healthcare facility in which a procedure is being operated. In such cases, the assignment or selection of audio channels or streams to various users may be managed by the healthcare facility, and adjusted or modified based on an authorization or approval provided by the healthcare facility or one or more entities managing the permissions associated with assigning and transmitting audio channels or streams to various users.

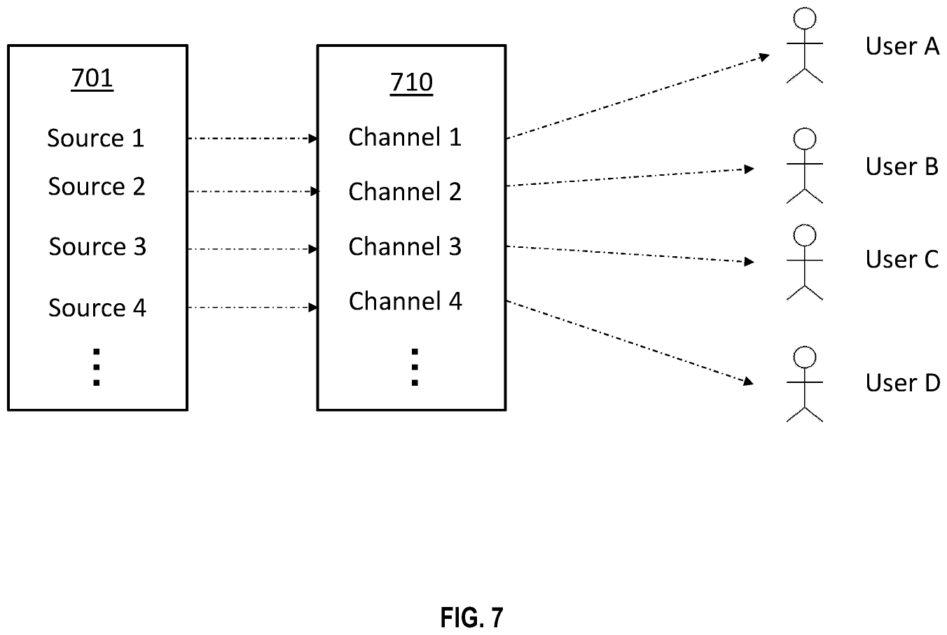

FIG. 7 schematically illustrates a plurality of audio sources 701 that are associated with a plurality of audio channels 710. The plurality of audio sources 701 may comprise, for example, source 1, source 2, source 3, source 4, and so on. The plurality of audio channels 710 may comprise, for example, channel 1, channel 2, channel 3, channel 4, and so on. The plurality of audio sources 701 may be mapped to one or more of the plurality of audio channels 710. The plurality of audio channels 710 may be automatically assigned to one or more users based on a function, a role, a specialty, an expertise, or an identify of the one or more users. The one or more users may have access to a subset of the plurality of audio channels 710. In some cases, different users may be able to connect to different audio channels. For example, user A may connect to audio channel 1 corresponding to audio source 1, user B may connect to audio channel 2 corresponding to audio source 2, user C may connect to audio channel 3 corresponding to audio source 3, and user D may connect to audio channel 4 corresponding to audio source 4. The assignment of users to specific channels or audio sources may be governed by the healthcare facility in which a procedure is being performed, by an administrator or an employee of the healthcare facility, or by a server or an entity managing one or more audio or data streams associated with the procedure.

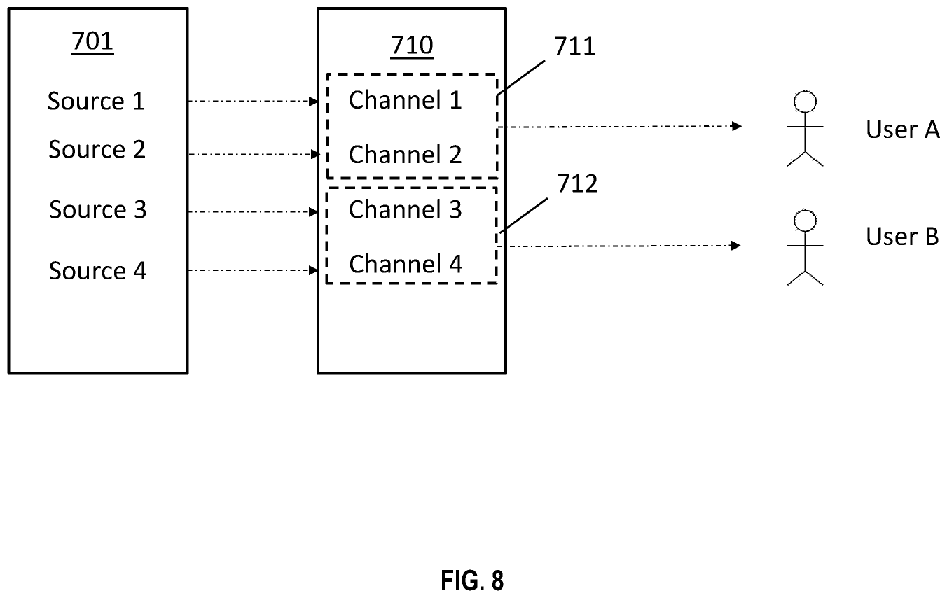

As shown in FIG. 8, in some cases the one or more users may select a particular audio channel or set of audio channels of interest. The selection of audio channels may directly correspond to a selection of one or more specific audio sources of interest. Alternatively, the selection of audio channels may be based on one or more parameters of interest (e.g., tool of interest, surgical phase of interest, medical technique of interest, surgeon or doctor of interest, etc.). In such cases, post-processing of surgical video and audio data may be performed to extract the audio sources of interest that correspond to the parameters of interest or the audio channels of interest selected by the one or more users. In some instances, user A may select a first group 711 of audio channels of interest and user B may select a second group 712 of audio channels of interest. The first group 711 of audio channels and the second group 712 of audio channels may correspond to different tools of interest, different surgical phases of interest, different medical techniques of interest, and/or different surgeons or doctors of interest.

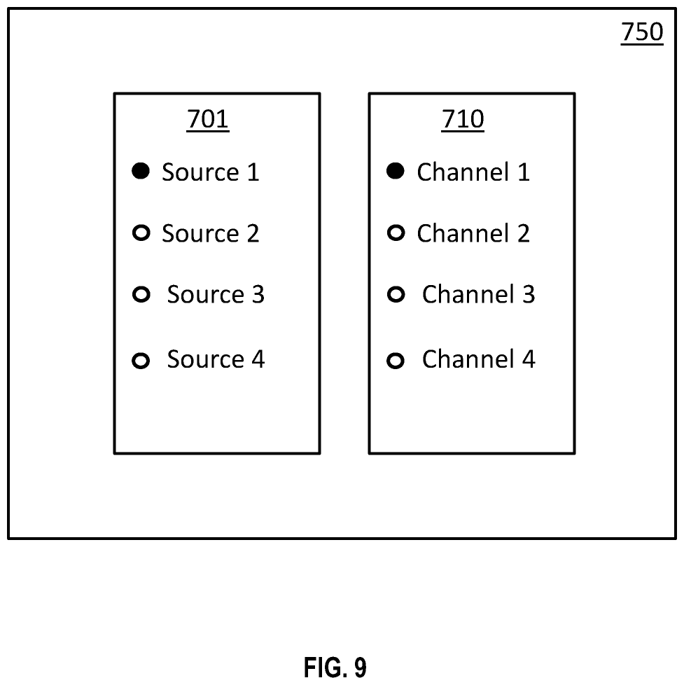

FIG. 9 schematically illustrates an example of a user interface 750 for selecting one or more audio sources or audio channels of interest from a plurality of audio sources 701 or audio channels 710. In some examples, a user may manually select the one or more audio sources 701 or audio channels 710 of interest by providing an input (e.g., a tap, a touch, a press, a click, etc.) to interact with a virtual element in the user interface 750. The virtual element may comprise, for example, a button, a checkbox, or a radio button. In some cases, the user interface 750 may permit the users to select a plurality of different audio channels or audio sources of interest at once.

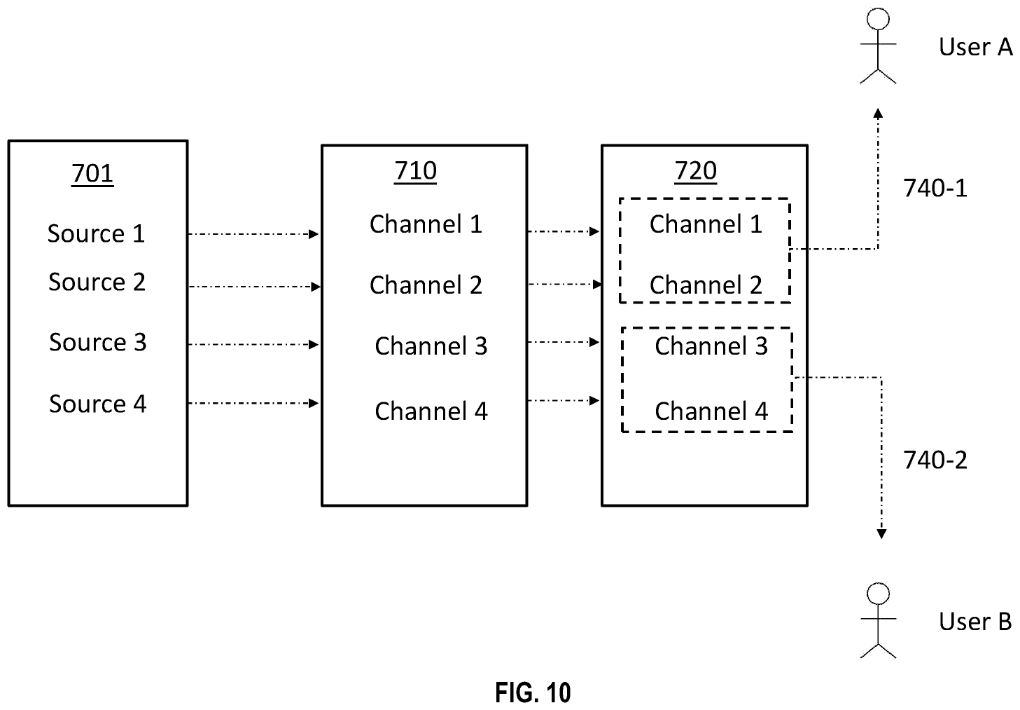

FIG. 10 schematically illustrates an audio management system 720 that is configured to perform post-processing of a plurality of audio sources 701 or audio channels 710 to provide a customized or tailored selection of audio channels to various users. The audio management system 720 may be implemented with aid of one or more processors. The audio management system 720 may be implemented on a computing device located at the healthcare facility or a server (e.g., a remote server or a cloud server). In some cases, the audio management system 720 may be configured to provide a first set of audio channels 740-1 to a first user B and a second set of audio channels 740-2 to a second user B. The audio management system 720 may be configured to select the first set of audio channels 740-1 and the second set of audio channels based on an identity, a role, an expertise, or a specialty of the user. In some cases, the audio management system 720 may be configured to select the first set of audio channels 740-1 and the second set of audio channels based on one or more inputs provided by the users. The one or more inputs may comprise, for example, a selection of one or more tools of interest, one or more surgical phases of interest, one or more medical techniques of interest, and/or one or more surgeons or doctors of interest.

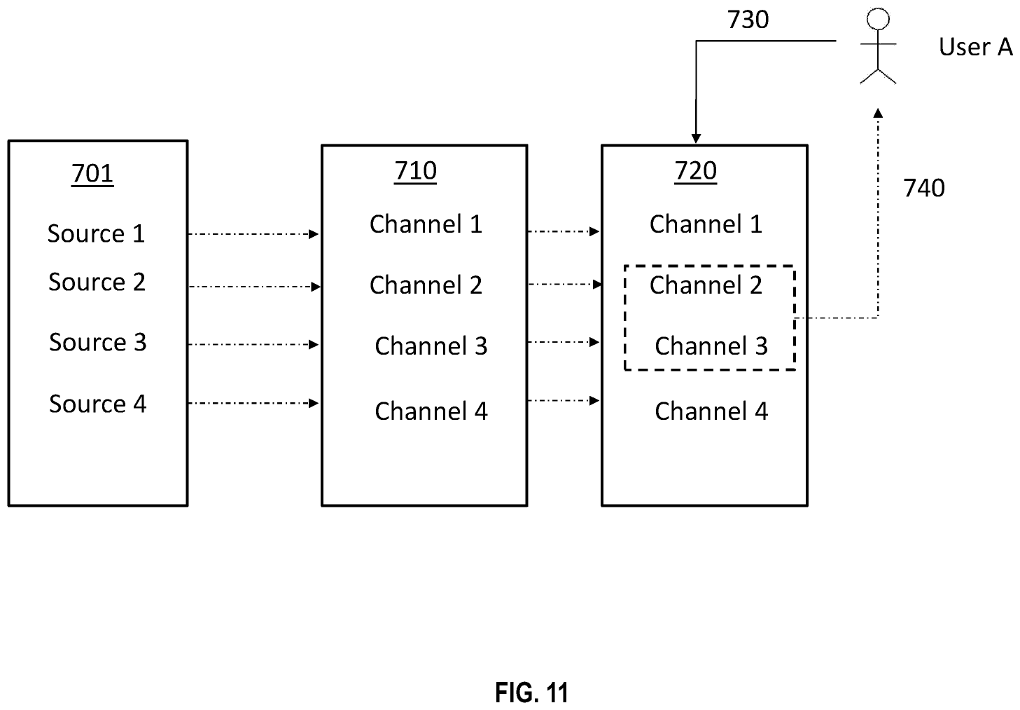

FIG. 11 schematically illustrates an audio management system 720 that is configured to adjust which audio channels are provisioned to a user, based on one or more inputs provided by the user. In some cases, a user may provide one or more inputs 730 to the audio management system 720. The one or more inputs 730 may comprise, for example, a selection of one or more tools of interest, one or more surgical phases of interest, one or more medical techniques of interest, and/or one or more surgeons or doctors of interest. The audio management system 720 may be configured to use the one or more inputs 730 to identify various channels of interest 740 for the user. The various channels of interest 740 may be associated with the one or more tools of interest, one or more surgical phases of interest, one or more medical techniques of interest, and/or one or more surgeons or doctors of interest indicated by the user. In some cases, a user may provide different inputs 730 at different times, and the audio management system 720 may be configured to adjust the selection of channels accordingly. The selection of channels may comprise audio data from different audio sources that correspond to the one or more inputs 730 provided by the user.

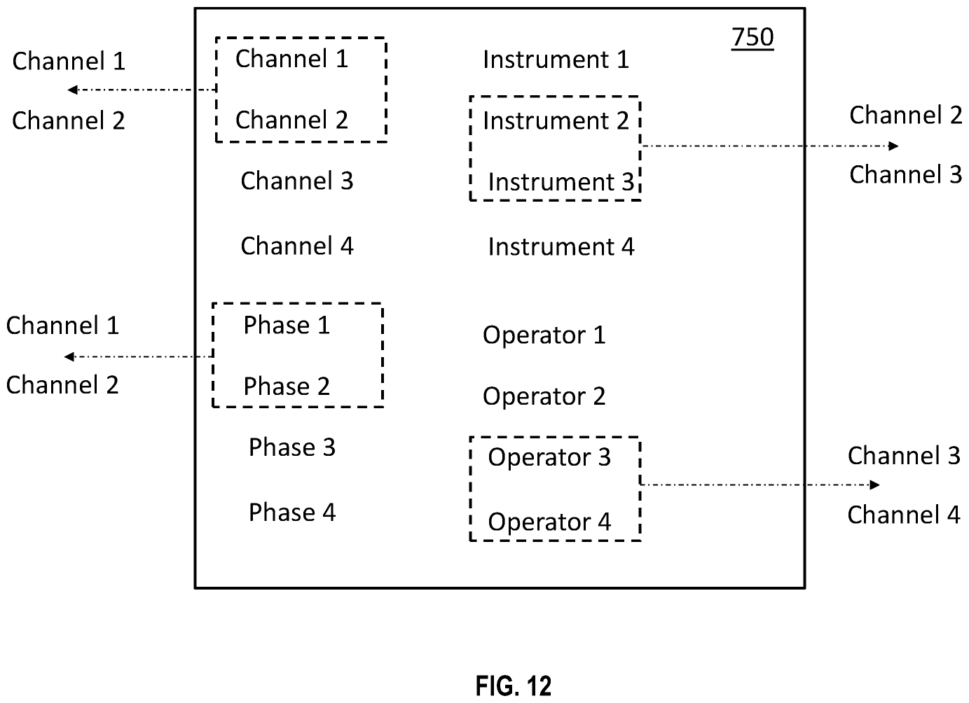

FIG. 12 schematically illustrates an exemplary user interface 750 for selecting various channels of interest. In some cases, a user may select one or more channels of interest, and the audio management system may be configured to provision one or more audio sources corresponding to the one or more channels of interest selected by the user. Such provisioning may involve post-processing of audio or video data to extract the relevant audio streams of interest, as described elsewhere herein. In some cases, a user may select various phases of interest, various instruments of interest, and/or various operators of interest. Based on such selections, the audio management system may be configured to provision one or more audio sources and/or one or more audio channels corresponding to the various parameters of interest selected by the user. In some embodiments, the user may make a plurality of selections corresponding to different instruments, phases, and operators of interest, and the audio management system may be configured to provision a plurality of audio sources and/or audio channels corresponding to the various selections made by the user.

In some cases, the audio channels of interest may change depending on the phase or stage of the surgical procedure. In some cases, one or more individuals viewing the surgical video may change the audio channels of interests or switch between two or more audio channels. In some cases, the one or more individuals viewing the surgical video may listen to two or more audio channels of interest simultaneously. In such cases, the audio channels may be associated with different features or aspects of the surgical procedure. For example, a first audio channel may be associated with a surgical tool or instrument, and the second audio channel may be associated with a surgeon or doctor using the surgical tool or instrument.

In some cases, the systems and methods of the present disclosure may be implemented to permit or enable audio collaboration among a plurality of individuals. In some cases, multiple individuals may simultaneously view a video of a surgical procedure. The video may comprise a live stream video or a recorded video. The individuals may separately select various audio beams or audio channels of interest and share a modified version of the surgical video with the audio beams or audio channels of interest with other individuals. In some cases, a first individual may modify the surgical video to include a first audio beam or channel of interest, and a second individual may further modify the surgical video to also include a second audio beam or channel of interest. In some cases, a third individual may view the surgical video containing both the first and second audio beams or channels, which surgical video may be shared to the third individual via a live stream or a through a server (e.g., a cloud server). The surgical video containing both the first and second audio beams or channels may provide the third individual with additional context with respect to various instruments, specialists, doctor, surgeons, views, or surgical phases associated with the surgical procedure.

In some cases, multiple remote vendors or specialists may provide audio commentary simultaneously to various portions or sections of a video of a surgical procedure. The audio commentary may comprise guidance, assistance, or an explanation, evaluation, or assessment of one or more steps or aspects of the surgical procedure. In some cases, a first individual may provide a first audio commentary and a second individual may provide a second audio commentary. The first audio commentary may be associated with a first audio channel and the second audio commentary may be associated with a second audio channel. In some cases, the surgical video containing the audio commentary from both the first and second individuals may be shared with a third individual. The surgical video may have the first audio channel comprising the first audio commentary and the second audio channel comprising the second audio commentary. In some cases, the surgical video containing both the first and second audio channels may allow various individuals viewing the surgical video to compare and contrast different approaches for performing the surgical procedure. In any of the embodiments described herein, the audio commentary by one or more users (e.g., remote vendors, specialists, surgeons, doctors, or medical workers) may be provided in place of or in addition to any audio streams or channels previously associated with the surgical video.

In some embodiments, one or more audio communications may be made during a surgical procedure. The one or more audio communications may comprise, for example, sounds made by an instrument (e.g., an ECG monitor or other medical hardware for monitoring various biological or physiological signals), a robot (e.g., a medical or surgical robotic system), or a human who is performing or assisting with the surgical procedure (e.g., one or more surgeons, doctors, nurses, assistants, and/or medical workers).

The audio communications made during a surgical procedure may be recorded and/or broadcasted to one or more users. In some cases, the audio communications may be recorded and broadcasted by a broadcaster (also referred to herein as a “publisher”). The audio communications may be broadcasted along with one or more images or videos of the surgical procedure.

In some cases, the broadcaster may broadcast the audio communications directly to a plurality of different users (e.g., one or more vendor representatives). Each of the plurality of different users may separately modify the audio communications broadcasted by the broadcaster. Modifying the audio communications may comprise, for example, selecting or enhancing various audio streams or audio channels of interest as described above, or eliminating or muting one or more audio streams or channels. In some cases, each individual may only modify the audio communications that he or she receives. For example, if a first user finds the beeping noises of an instrument distracting or annoying, the first user can mute the audio streams or channels associated with such beeping noises, without modifying the audio streams or channels broadcasted to a second user (who may be interested in monitoring the beeping noises that the first user found to be distracting and annoying). In other cases, each individual may modify the audio communications for other individuals or users receiving the audio communications from the broadcaster. For example, if a user finds the beeping noises of an instrument to be distracting or annoying, and the user believes that other users would also find the beeping noises distracting or annoying, the user can mute the audio streams or channels associated with such beeping noises for various other users (e.g., as a preemptive measure or a courtesy for other users). The systems and methods of the present disclosure may be implemented to allow each individual user to mute specific channels for themselves, or alternatively, for all other participants receiving the audio communications from the broadcaster. In some cases, the systems and methods of the present disclosure may also be implemented to allow individual users to modify, enhance, or tune specific channels for themselves and/or other participants receiving the audio communications from the broadcaster.

In some cases, the broadcaster may broadcast the audio communications to a moderating entity (e.g., a human or a server). The moderating entity may be configured to receive and pre-process or modify the audio communications before they are broadcasted to the one or more users. For example, the moderating entity may enhance certain audio communications of general interest, and/or mute or eliminate other audio communications that are of less interest or importance. In some cases, the moderating entity may mute or eliminate certain audio communications that reveal personal or private information, or audio communications that are distracting or annoying. The audio communications modified by the moderating entity may be transmitted to one or more users, who may further modify the audio communications to their respective preferences. In some cases, the moderating entity may pre-process or modify the audio communications broadcasted by the broadcaster in different ways for different users or subsets of users. For example, the moderating entity can enhance and/or eliminate a first set of audio channels for a first subset of users, and enhance and/or eliminate a second set of audio channels for a second subset of users. In either case, the first and second subset of users may further tune the audio communications they receive based on individual needs and/or preferences.

In some cases, the broadcaster may modify the audio communications broadcasted to the one or more users and/or the moderating entity between the broadcaster and the one or more users. As described above, modifying the audio communications may comprise selecting or enhancing various audio streams or audio channels of interest, or eliminating or muting one or more audio streams or channels. The moderating entity and/or the one or more users may make further modifications to the audio communications modified by the broadcaster. In some cases, the broadcaster may enhance and/or eliminate different audio channels for different subsets of users, based on an identity, a role, an expertise, or a specialty of the users. The broadcaster may control which audio channels or streams are broadcasted to the moderating entity or the one or more users.

In some cases, each individual user, viewer, moderator, or remote specialist can choose which audio streams to be enhanced or eliminated. In some cases, each individual user, viewer, moderator, or remote specialist can choose which audio streams to be enhanced or eliminated for all participants. In other cases, each individual user, viewer, moderator, or remote specialist can only modify the audio streams that he or she has received, is receiving, or will receive.

Audio tuning may be performed by the broadcaster, the remote vendor representatives, and/or individual viewers. If the audio is not clear for any reason (e.g., due to ambient noise or other auditory disturbances), the audio may be tuned to individual preference. In some cases, the audio may be tuned automatically using one or more audio optimization algorithms. In other cases, the audio may be tuned manually by the one or more users. Audio tuning may comprise, for example, increasing or decreasing a volume of one or more audio communications, speeding up or slowing down one or more audio channels, changing a pitch, a tone, a timber, a rhythm, or a bass level of one or more audio communications, filtering out various frequencies or ranges of frequencies, or otherwise modifying the actual audio signals. In some cases, the audio tuning may be used to reduce ambient noise, static, reverberations, and/or echoes that are present when listening to the audio communications. In some cases, the audio tuning may comprise boosting certain audio signals or certain frequencies of the audio signals to improve the intelligibility of words and to decrease the tiredness of viewers and listeners.

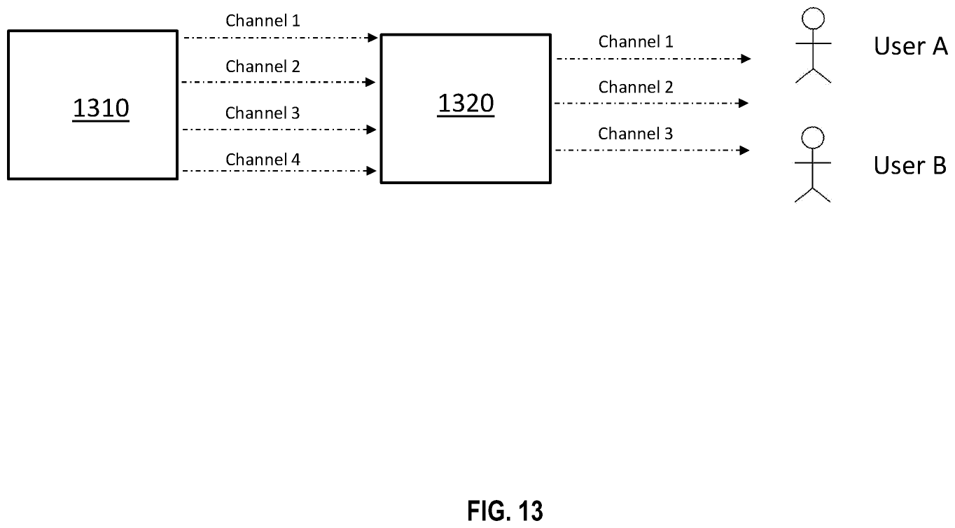

FIG. 13 schematically illustrates a broadcaster 1310 configured to broadcast one or more audio channels. The broadcaster 1310 may broadcast a plurality of audio channels (e.g., channel 1, channel 2, channel 3, and channel 4) to a moderator entity 1320. In some cases, the broadcaster 1310 may select a particular subset of audio channels to transmit to the moderating entity 1320. The moderating entity 1320 may be configured to enhance one or more of the audio channels before the audio channels are transmitted to one or more users or viewers. The moderating entity 1320 may be configured to mute one or more of the audio channels received from the broadcaster 1310. For example, the moderating entity 1320 may receive a plurality of channels (e.g., channel 1, channel 2, channel 3, and channel 4) from the broadcaster 1310 and transmit a subset of the plurality of channels (e.g., channel 1, channel 2, and channel 3) to user A and user B.

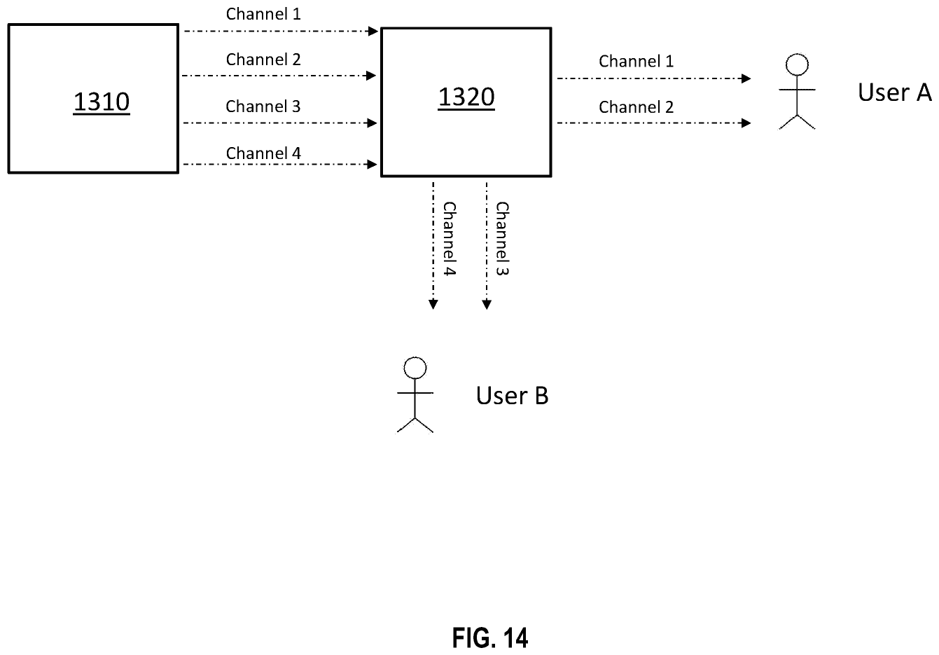

FIG. 14 schematically illustrates a broadcaster 1310 configured to broadcast one or more audio channels. The broadcaster 1310 may broadcast a plurality of audio channels (e.g., channel 1, channel 2, channel 3, and channel 4) to a moderator entity 1320. The moderating entity 1320 may be configured to selectively transmit a first subset of audio channels (e.g., channel 1 and channel 2) to a first user and a second subset of audio channels (e.g., channel 3 and channel 4) to a second user. In some cases, the moderating entity 1320 may be configured to selectively enhance or mute certain audio channels for certain users (e.g., based on user preference, user identity or expertise, or based on one or more permissions granted to various users) before transmitting the modified audio communications to the users.

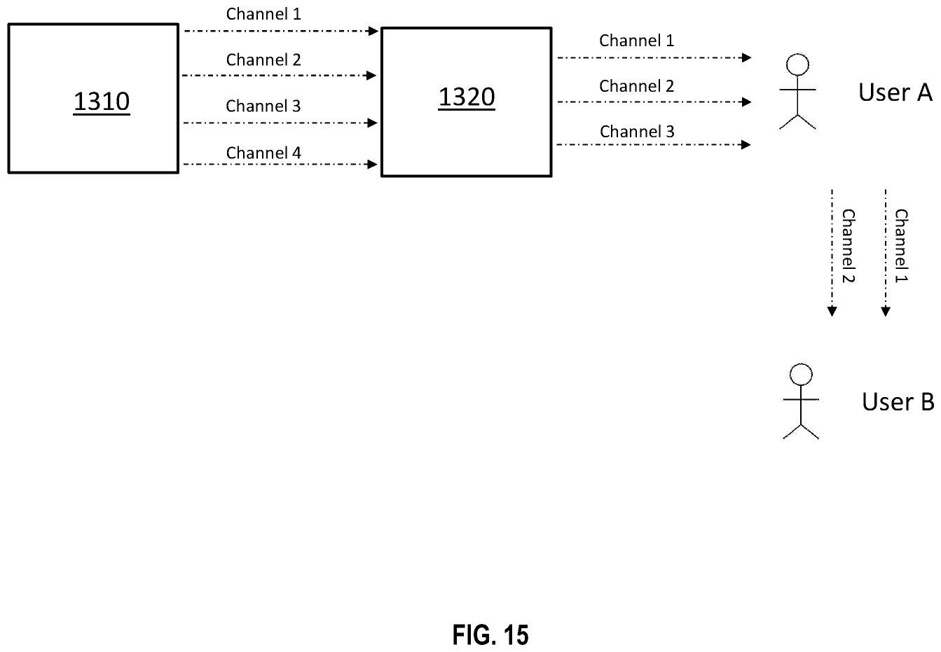

FIG. 15 schematically illustrates a broadcaster 1310 configured to broadcast one or more audio channels. The broadcaster 1310 may broadcast a plurality of audio channels (e.g., channel 1, channel 2, channel 3, and channel 4) to a moderator entity 1320. The moderating entity 1320 may be configured to selectively transmit a subset of the audio channels (e.g., channel 1, channel 2, and channel 3) to a first user (e.g., user A). The first user may be, for example, a remote vendor representative or a remote specialist. The first user may enhance, eliminate, and/or modify one or more of the audio channels received from the moderating entity 1320. In some cases, the first user may forward or rebroadcast a second subset of the audio channels (e.g., channel 1 and channel 2) to a second user (e.g., user B). The second user may be, for example, another remote vendor representative or remote specialist. Alternatively, the second user may be any listener or viewer who is interested in receiving and listening to one or more modified or enhanced audio communications associated with a surgical procedure. For example, the second user may be a doctor, a surgeon, a medical assistant, a medical worker, a friend or family member of the patient, a medical student, a medical resident, or an intern. In some cases, the second user may further tune the audio channels received from the first user based on the second user's needs or preferences.

In some embodiments, the microphone arrays of the present disclosure (also referred to herein as mic arrays, mic array modules, or microphone array modules) may comprise one or more cameras or image sensors. The one or more cameras or image sensors may have a field of view spanning an area in which audio signals can be captured or detected using one or more microphones of the mic array module. The cameras or image sensors may be used to capture one or more images or videos of one or more audio sources from which one or more detectable audio signals originate. The one or more audio sources may comprise, for example, a doctor, a surgeon, a medical worker, an assistant, a tool (e.g., a medical tool), an instrument, or a device.

In some embodiments, the one or more images or videos can be sent out to one or more remote participants so that the remote participants can view (1) the audio source associated with one or more audio signals detected or captured using the mic array module, or (2) an area in a surgical environment in which the one or more audio signals are detected. In some cases, the view of the audio source or the area in which the one or more audio signals are detected can be displayed to various remote participants in real time as the one or more audio signals are detected. In some cases, different remote participants may be provided different fields of view corresponding to different audio sources or different sets of audio signals of interest.

In some embodiments, a remote participant may select (1) which audio beams the remote participant would like to pick up and/or (2) which field of view the remote participant would like to investigate or monitor. The field of view may correspond to an area or region from which one or more audio beams of interest can originate. In some cases, the remote participant may also select or specify one or more audio beams of interest, one or more audio sources of interest, or one or more regions of interest. In some cases, the regions of interest may correspond to an area or an environment in which the one or more audio sources are located. In some cases, the selection of audio beams of interest, audio sources of interest, and/or regions of interest may be performed locally or remotely.

In some embodiments, the mic array module may comprise one or more cameras or image sensors. The one or more cameras or image sensors may provide users with a field of view of a surgical environment. The field of view may be used to visually tag doctors, nurses, vendor representatives, remote specialists, local specialists, and/or anyone participating in, supporting, or monitoring a procedure performed in the surgical environment, either locally in the surgical environment or remotely at a location that is remote from the surgical environment. In some cases, the field of view may also enable users to specify if they are interested in a person's audio signals or if the user would like to specify removal or filtering of that person's audio signals. In some cases, the mic array module may also track one or more individuals within the field of view of the one or more cameras or imaging sensors and adjust audio beams or the field of view (which may correspond to one or more regions of interest) as the individual moves within the surgical environment. The adjustment of the audio beams, the field of view, or the region of interest to be monitored may be performed using software and/or by physically changing a position and/or an orientation of the mic array module or any components thereof.

In some embodiments, a selection of various audio signals of interest, audio sources of interest, or regions/fields of view of interest can be pre-registered, pre-determined, or pre-programmed before a procedure occurs. The selection may be adjustable by users (e.g., before, during, and/or after the procedure) based on personal user preference or previous selections made by the user (or other users) for similar procedures. In some cases, selections of various audio signals of interest, audio sources of interest, or regions/fields of view of interest can be made on recorded content or live content, and users can then select which subset of audio signals they are interested in (and/or not interested in). In some cases, the audio signals of interest may be further enhanced as described elsewhere herein. In some cases, the audio signals which are not of interest may be muted, attenuated, or otherwise filtered out so that a user or participant (e.g., a remote participant) can focus on the audio signals of interest.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for enhancing audio communications, comprising:
(a) detecting one or more audio communications associated with a medical procedure and one or more parameters associated with the one or more audio communications; and
(b) processing the one or more audio communications based on the one or more parameters to generate one or more enhanced audio communications.

2. The method of claim 1, wherein the one or more parameters comprise a physical feature, a face, a voice, or an identity of a human or a robot that made the one or more audio communications.

3. The method of claim 1, wherein the one or more parameters comprise a key word, phrase, or sentence of the one or more audio communications.

4. The method of claim 1, wherein the one or more parameters comprise a type of tool or instrument in use or a phase of the medical procedure.

5. The method of claim 1, wherein processing the one or more audio communications comprises beam forming to adjust a detection area, a detection range, a directivity, or a directionality of one or more audio detection devices.

6. The method of claim 1, wherein processing the one or more audio communications comprises prioritizing a detection or a capture of the one or more audio communications based on an identity of a speaker.

7. The method of claim 6, wherein processing the one or more audio communications comprises adjusting the priority of detection or capture based on a detection of one or more key words, phrases, or sentences in the one or more audio communications.

8. The method of claim 1, wherein processing the one or more audio communications comprises increasing a volume of a first audio communication of the one or more audio communications relative to a volume of a second audio communication of the one or more audio communications.

9. The method of claim 1, wherein processing the one or more audio communications comprises decreasing a volume of a first audio communication of the one or more audio communications relative to a volume of a second audio communication of the one or more audio communications.

10. The method of claim 1, wherein processing the one or more audio communications comprises muting or eliminating one or more audio communications.

11. The method of claim 1, wherein the one or more enhanced audio communications correspond to a tool or instrument of interest or a usage of the tool or instrument of interest.

12. The method of claim 1, wherein the one or more enhanced audio communications correspond to a surgical phase of interest.

13. The method of claim 1, wherein the one or more enhanced audio communications correspond to a doctor, a surgeon, a medical worker, a vendor representative, or a product specialist of interest.

14. The method of claim 1, further comprising detecting the one or more parameters using computer vision, natural language processing, or machine learning.

15. The method of claim 1, wherein detecting the one or more parameters comprises identifying a medical tool or instrument that is associated with the one or more audio communications.

16. The method of claim 15, wherein identifying the medical tool or instrument comprises imaging the tool or instrument, scanning a identifier associated with the tool or instrument, or receiving one or more electromagnetic waves comprising information on the tool or instrument.

17. A method for enhancing audio communications, comprising:
(a) receiving a plurality of audio communications associated with a medical procedure;
(b) receiving one or more user inputs corresponding to a parameter of interest, wherein the parameter of interest is associated with a performance of one or more steps of the medical procedure; and
(c) generating one or more enhanced audio communications based on the plurality of audio communications and the one or more user inputs.

18. The method of claim 17, wherein the one or more user inputs comprise a user selection of the parameter of interest.

19-30. (canceled)

31. The method of claim 1, wherein processing the one or more audio communications comprises (i) enhancing one or more audio communications or (ii) muting or eliminating one or more audio communications for one or more users.

32. The method of claim 31, wherein the one or more audio communications are processed by a broadcaster, a moderating entity, a remote specialist, a vendor representative, or the one or more users, wherein the one or more users comprise at least one user viewing a surgical video or a portion thereof.

33-54. (canceled)
